(19) United States
(12) Patent Application Publication
(10) Pub. No.: US 2007/0141034 A1
(43) Pub. Date: Jun. 21, 2007

(54) Methods for isolating t cells and uses thereof

(76) Inventors: R. Paul Johnson, Lexington, MA (US); George B. Cohen, Charlestown, MA (US)

Correspondence Address: FOLEY HOAG, LLP;PATENT GROUP (w/HUV HMV), 155 SEAPORT BLVD., BOSTON, MA, 02210-2600, US

(21) Appl. No.: 10/565,359

(22) PCT Filed: Jul. 23, 2004
(86) PCT No.: PCT/US04/23759

§ 371 Date: Dec. 26, 2006

Related U.S. Application Data
(60) Provisional application No. 60/489,557, filed on Jul. 23, 2003.

(51) Int. Cl. C12N 5/08; A61K 35/14

(52) U.S. Cl. 424/93.7; 435/372

(57) ABSTRACT

Provided herein are methods for obtaining viable populations of T cells and enriched populations of T cells.

### GOVERNMENT SUPPORT

This invention was made with government support under grant Nos. AI45314, RR00168 and AI43890 from the National Institutes of Health. The government has certain rights in the invention.

### BACKGROUND

T lymphocytes, or T cells, function as the body's primary means of recognizing antigenic determinants on the cell surface. Interactions between specific ligands on the surface of the T cell and a cell displaying or presenting the antigen lead to T cell activation, which in turn leads to a variety of immunogenic responses, including cytokine synthesis and release, T and natural killer (NK) cell-mediated cytotoxicity, and activation of macrophages and B cells.

The course of T cell differentiation is crucial to the outcome of an immune response. Early in this process, T cells are committed to develop into one of several functionally distinct subsets, including Th1, Th2, and the recently described T regulatory (Tr) cells (Jonuleit et al., 2000, J. Exp. Med. 192:1213-1222).

T cell differentiation is regulated by the local microenvironment. Hence, the property of antigens (Ags) encountered by the T cell, and the expression of costimulatory molecules and cytokines by antigen presenting cells (APCs) strongly influence T cell differentiation. The stimulated T cell undergoes a sequence of phenotypic changes beginning with its progression from the resting state to mitosis and later to differentiation into effector and memory cells. Among the earliest (immediate) changes, observable within 15-30 minutes of stimulation, are the expression of genes encoding transcription factors such as c-Fos, NF-AT, c-Myc and NF-kappaB, protein kinases such as Jak-3 and protein phosphatases such as Pac-1. The subsequent early changes, occurring within several hours of stimulation, mark the beginning of the expression of genes encoding activation markers. These include several cytokines (IL-2 and others), IL-2 receptor subunit alpha (CD25), insulin receptor, transferrin receptor and several other surface molecules such as CD40L, CD 26, CD30, CD54, CD69 and CD70.

Activation markers reach a maximum level of expression just before the first division, 24 hours after stimulation. During this period the level of expression of several other molecules already expressed on resting T cells increases. At a later point, some days after activation commenced, late activation antigens become expressed on the T cells. These include MHC class II molecules and several members of the beta 1 integrin family. Expression of late activation antigens marks the differentiation of the activated cell into effector or memory T cells.

T cells play important roles in autoimmunity, inflammation, cytotoxicity, graft rejection, allergy, delayed-type hypersensitivity, IgE-mediated hypersensitivity, and modulation of the humoral response. Disease states can result from the activation of self-reactive T cells, from the activation of T cells that provoke allergic reactions, or from the activation of autoreactive T cells following certain bacterial and parasitic infections, which can produce antigens that mimic human protein, rendering these protein “autoantigens”. These diseases include, for example, the autoimmune diseases, autoimmune disorders that occur as a secondary event to infection with certain bacteria or parasites, T cell-mediated allergies, and certain skin diseases such as psoriasis and vasculitis. Furthermore, undesired rejection of a foreign antigen can result in graft rejection or even infertility, and such rejection can be due to activation of specific T lymphocyte populations. Pathological conditions can also arise from an inadequate T cell response to a tumor or a viral infection. In these cases, it would be desirable to increase an antigen-specific T cell response in order to reduce or eliminate the tumor or to eradicate an infection.

In the treatment of cancers, cellular immunotherapy has been employed as an alternative, or an adjunct to, conventional therapies such as chemotherapy and radiation therapy. For example, cytotoxic T lymphocyte (CTL) responses can be directed against antigens specifically or preferentially presented by tumor cells. Following activation with T cell cytokines in the presence of appropriately presented tumor antigen, tumor infiltrating lymphocytes (TILs) proliferate in culture and acquire potent anti-tumor cytolytic properties (Weidmann et al. (1994) Cancer Immunol. Immunother. 39:1-14).

The introduction into a cancer patient of in vitro activated lymphocyte populations has yielded some success. Adoptive immunotherapy, the infusion of immunologically active cells into a cancer patient in order to effect tumor regression, has been an attractive approach to cancer therapy for several decades. Two general approaches have been pursued. In the first, donor cells are collected that are either naturally reactive against the host's tumor, based on differences in the expression of histocompatibility antigens, or made to be reactive using a variety of “immunizing” techniques. These activated donor cells are then transfused to a tumor-bearing host. In the second general approach, lymphocytes from a cancer patient are collected, activated ex vivo against the tumor and then reinfused into the patient. Triozzi (1993) Stem Cells 11:204-211; and Sussman et al. (1994) Annals Surg Oncol. 1:296.

Current methods of cancer treatment are relatively non-selective. Surgery removes the diseased tissue, radiotherapy shrinks solid tumors and chemotherapy kills rapidly dividing cells. Systemic delivery of chemotherapeutic agents, in particular, results in numerous side effects, in some cases severe enough to preclude the use of potentially effective drugs.

Viral diseases are also candidates for immunotherapy. Heslop et al. (1996) Nature Med. 2:551-555. Immunological responses to viral pathogens are sometimes ineffective in eradicating or sufficiently depleting the virus. Furthermore, the highly mutable nature of certain viruses, such as human immunodeficiency virus, allows them to evade the immune system.

Over the past several years, a number of approaches have been developed for the identification of antigen-specific T cells on a single-cell basis. These techniques have included ELISPOTs, intracellular cytokine staining (ICS) and MHC tetramers or dimers. These techniques have numerous advantages as compared with traditional assays of cell-mediated immunity, including improved sensitivity, quantitation, and the ability to provide phenotypic data on antigen-specific cells identified by tetramers or ICS. However, these approaches have important limitations as well.

For example, intracellular cytokine staining (Picker et al., 1995), requires chemical fixation of cells prior to staining, and therefore is not suitable for cellular RNA analysis (Masuda et al., 1999) or for isolation of cells for adaptive transfer experiments. Moreover, the effector and memory T cells that persist in wake of a viral challenge are a heterogeneous population, differing in their gene expression profiles, homing patterns, and functional capabilities, such as the ability to secrete cytokines (Abbas et al., 1996; Butcher and Picker, 1996; Lanzavecchia and Sallusto, 2000; McKay et al., 2002; Welsh, 2001). Therefore, a secretion assay which isolates live antigen-specific T cells based on a particular cytokine, e.g. IFN-γ, may not detect all antigen-specific cells and there is a need to complement existing assays with alternative approaches. Finally, although MHC multimers have revolutionized the study of antigen-specific CD8+ T cells (Altman et al., 1996; Dal Porto et al., 1993), MHC multimers have not met the same success when applied to CD4+ T cells (Hackett and Sharma, 2002). Partly, this reflects the lower frequency of epitope-specific CD4+ T cells compared to CD8+ T cells (Whitmire and Ahmed, 2001), and partly it reflects that engineering a MHC class II multimer is a more challenging task than it is for class I (Hackett and Sharma, 2002).

### SUMMARY

Provided herein are methods for isolating a T cell population, comprising (i) contacting a population of cells comprising a T cell with a first activator that binds to a T cell receptor on the T cell, thereby activating the T cell and a first agent that binds to a first cell surface molecule on the T cell, to obtain a T cell population bound by the first agent; and (ii) isolating the T cell population by a method using the first agent. The T cell may be a CD4+ T cell.

The first cell surface molecule may be an activation marker, e.g., CD40L or CTLA-4. In one embodiment, the first agent is an antibody or portion thereof sufficient for binding specifically to the surface molecule. The first agent may be labeled directly or indirectly.

Methods of using the first agent include fluorescence activated cell sorting (FACS). In another embodiment, the methods of using the first agent comprise using a solid surface to which the T cell binds. When the first agent is not labeled, it may be detected with a first detection agent that specifically binds to the first agent. The first detection agent may be labeled.

The first activator may bind to the antigen-binding region of the T cell receptor. For example, the first activator may be an antigen that may be located on an antigen presenting cell. In another embodiment, the first activator does not bind to the antigen-binding region of the T cell receptor. For example, the first activator may be a superantigen or a polyclonal activator.

The population of cells may be obtained from any mammal, e.g., a human or a non-human primate. For example, human cells may be obtained from a patient. The population of cells from which a T cell population may be isolated may comprise peripheral blood mononuclear cells or may comprise bone marrow cells.

The population of cells comprising a T cell may be contacted essentially simultaneously with a first agent and a first activator. In one embodiment, the population of cells comprising a T cell is contacted with a first agent prior to being contacted with a first activator, wherein the T cell is contacted simultaneously with the first activator and the first agent for at least about 10 minutes. The population of cells comprising a T cell may also be contacted with a first activator prior to being contacted with a first agent, wherein the T cell is contacted simultaneously with the first activator and the first agent for at least about 10 minutes. Methods may further comprise contacting the population of cells comprising a T cell with a first agent after contacting the T cell with a first activator.

Methods of isolating a T cell population may further comprise (i) contacting the T cell population with (a) a second activator that binds to the T cell receptor on at least some cells of the T cell population thereby activating at least some cells of the T cell population and (b) a second agent that binds to a second cell surface molecule of at least some cells of the T cell population, to obtain a T cell population bound by the second agent; and (ii) isolating the T cell population by a method using the second agent. The second activator may be the same or may be different from the first activator. The second agent may be the same of may be different from the first agent. The second cell surface molecule may be the same or may be different from the first cell surface molecule. For example, the first cell surface molecule may be CD40L and the second cell surface molecule may be CTLA-4.

Also provided herein are isolated viable cell populations, wherein at least about 90% of the cell population consist of viable T cells. The T cells may be human or non-human primate cells. At least 90% of the cell population provided herein may consist of viable CD4+ T cells, CD40L+CD4+ T cells or CTLA-4+CD4+ T cells. Further provided is an isolated viable T cell population isolated by (i) contacting a population of cells comprising a T cell with a first activator that binds to a T cell receptor on the T cell thereby activating the T cell and a first agent that binds to a first cell surface molecule on the T cell, to obtain a T cell population bound by the first agent; and (ii) isolating the T cell population by a method using the first agent.

Also provided herein are methods of treating a subject having a disease, e.g., cancer or an infectious disease, comprising (i) obtaining a population of cells comprising a T cell from the subject; (ii) subjecting the population of cells to the methods described herein to thereby obtain a T cell population; and (iii) administering the T cell population to the subject.

The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE DRAWINGS

FIG. 1 shows the upregulation of Activation Markers on Antigen-Stimulated Cells. PBMC were stimulated with (columns left to right): control, superantigen, or whole CMV antigen and stained for CD69 (y-axis) and: (A) TNFα by ICS; (B) CD40L by ICS; (C) CD40L by surface staining after antigen stimulation; or (D) CD40L by surface staining during antigen stimulation. Except where noted for CD40L, the cells were stained for other cell surface markers: CD3, CD4, and CD69 after antigen stimulation. Cells shown are gated on CD3+CD4+ lymphocytes. Percentages shown refer to the indicated highlighted population. Cells were antigen stimulated for ˜7 hours. This time frame is optimal for both CD40L expression and TNFα production. CD69-APC surface staining (C & D) was consistently brighter than CD69-APC ICS staining (B). CD69-staining in FIG. 1A was done with a different fluorophore, CD69-PE, and is therefore not comparable to the other three CD69-APC stains. All flow cytometry graphs presented in this manuscript are based on a logarithm scale.

FIG. 2 is a time course for CD40L Surface Expression. PBMC were stimulated with superantigen and stained for CD40L either: during antigen stimulation (squares) or after antigen stimulation (circles). CD40L expression was determined in CD3+CD4+CD69+ gated cells as described in FIG. 1C & D.

FIG. 3 shows surface staining for CD25 and CTLA-4. PBMC were stimulated with superantigen and stained for (A) CD25 or (B) CTLA-4 either after antigen stimulation (left-column) or during antigen stimulation (right-column). Cells shown are gated on CD3+CD4+ lymphocytes. Percentages refer to the indicated highlighted population. The number shown in italic to the right of the percentage, indicates the MFI over background for either CD25 or CTLA-4 in the highlighted population. All CD69 staining in FIG. 3 followed antigen stimulation. Because CTLA-4 and CD25 expression kinetics are slower than that of CD40L (FIG. 2) flow analysis in FIG. 3 followed 15 hours of antigen stimulation.

FIG. 4 demonstrates results of low pH washing of CD40L and CTLA-4 stained cells. PBMC were stimulated with superantigen and stained for CD40L (A & B) or CTLA-4 (C & D) either after antigen stimulation (A & C) or during antigen stimulation (B & D). Following CD40L or CTLA-4 surface staining the cells were: analyzed immediately (left column); acid washed (middle column); or acid washed and then restained for either CD40L or CTLA-4 (right column). Cells shown are gated on CD3+CD4+ lymphocytes. The number shown in italic indicates the MFI over background for either CD40L or CTLA-4 in the indicated population. Except for CD40L and CTLA-4 staining, all other staining (CD3, CD4, and CD69) followed antigen stimulation and acid treatment. Prior acid treatment of cells only slightly affected subsequent staining for CD3, CD4 and CD69.

FIG. 5 shows magnetic bead purification of CMV-specific CD4+ T cells. PBMC were stimulated with either: (A) control or (B) CMV antigen and CD40L-PE stained during antigen stimulation. The cells in (B) were then incubated with anti-PE magnetic beads and passed over a magnetic column. Flow-through from the column is shown in (C) and column eluted cells in (D). The cells in (D) were expanded in culture for 12 days and then incubated with autologous B cells (2:1 B:T cell ratio) pulsed with either: (E) control or (F) CMV antigen. Cells shown are gated on CD3+CD4+ lymphocytes. Percentages refer to the indicated population.

FIG. 6 demonstrates the genetic Modification of CD4+CD40L+ T cells. PBMC were stimulated with superantigen and CD40L-PE stained during stimulation. CD40L+ T Cells were enriched using magnetic beads (as in FIG. 5) and further purified by fluorescence activated cell sorting and expanded in vitro. After five days in culture the CD40+ enriched cells were transduced with various GFP encoding retroviral constructs. Shown are the cells at one week (A, B, & D) and twelve weeks (C & E) following transduction with the following promoter/retroviral constructs: (A) Control Cells; (B & C) MPSV promoter-GFP/lentiviral vector; (D & E) long-terminal repeat promoter-GFP/MFG murine retroviral vector. Percentages shown refer to the indicated population. The number shown in italic below the percentage indicates the MFI of GFP+ cells.

FIG. 7 shows the effect of Concanavalin A Stimulation on GFP Expression. Three polyclonal rhesus T cell lines were transduced with the GFP encoding MFG murine retroviral vector (as in FIG. 6D) and GFP expression monitored over time. At week seven, one week following the last Concanavalin A restimulation, the cell lines were split and either: restimulated with Concanavalin A and irradiated feeder cells (squares) or maintained in media/IL-2 without restimulation (circles). GFP expression was monitored over the next week. Shown is the average GFP expression for the three cell lines normalized to GFP expression prior to Concanavalin A treatment. Individually, GFP expression in all three cell lines peaked 6 days following Concanavalin A treatment and was up: 190%, 240%, and 300%, respectively, over media/IL-2 treated cells (average increase=245%).

FIG. 8. Superantigen activation of CD4 T cells leads to TNFα, CD69, and CD40L upregulation through TCR-dependent pathways. Human PBMC were stimulated with either: SEE (left-hand column) or SEB (right-hand column) for 7 hours and stained for Vβ17+ (x-axis) and: TNFα (top row), CD69 (middle row), or CD40L (bottom row). Percentages shown indicate the percentage of Vβ17-negative or Vβ17-positive cells that lie above the horizontal line. Hence, for SEE stimulated CD4 T cells (top left-hand graph), 5.0% of Vβ17-negative cells are TNFα+, while 0.1% of Vβ317-positive cells are TNFα+, etc.

FIG. 9. Comparison of the relative ability of naïve, TCM, and TEM cells to secrete cytokines and upregulate surface activation markers. (A) Rhesus PBMC were stimulated with superantigen for 7 hours and stained for cell surface expression of CD28 and CD95 and (top row) intracellular TNFα, or (bottom row) CD69 surface expression. Shown are the CD28 and CD95 staining profile for: i) all CD4 T cells, ii) only TNFα+ CD4 T cells, iii) all CD4 T cells, and iv) only CD69+ CD4 T cells. Gates for naïve, TCM, and TEM are as indicated. Neither CD28 nor CD95 surface expression was significantly altered during the 7 hour assay (data not shown). (B) Rhesus PBMC were stimulated with SEA for 7 hours and the ability of naïve (first column), TCM (second column), or TEM (third column) CD4 T cells to upregulate: CD69 (blue squares), CD40L (red squares), INFα (dark green circles), IFNγ (aqua green circles), or IL-2 (purple circles) measured. Classification of rhesus CD4 T cells as naïve, TCM, or TEM is as shown in FIG. 7A. PBMC were SEA stimulated with costimulation provided by plate-immobilized anti-CD49d antibody. Similar results were seen in a more limited study where costimulation was provided by anti-CD49d and anti-CD28 antibodies, however, the presence of the CD28 antibody made later staining for CD28 for flow cytometric analysis less reliable. Although for the animal shown here, CD69 upregulation was greater in naïve and TCM cells than it was in TEM cells, this was not generally true. The results shown are typical of the 7 macaques tested for TNFα secretion and 5 macaques tested for IFNγ and IL-2 secretion. Qualitatively similar results were seen over a 7-24 hour time course. Superantigen stimulation did not lead to significant secretion of the one Th2 cytokine tested (IL-4).

FIG. 10. Comparison of the relative ability of Naive, TCM, and TEM cells to secrete TNFα, IFNγ, or IL-2. Rhesus PBMC were stimulated with SEA and SEB for 7 hours and stained for CD4, CD28, TNFα and (top row) IFNγ, or (bottom row) IL-2. CD28-negative CD4 T lymphocytes were classified as TEM as in FIG. 9A. However, both naïve and TCM are CD28+ (FIG. 9A) and the use of two fluorophores for cytokine analysis precludes costaining with CD95 to distinguish naïve and TCM cells (as was done in FIG. 9A). Therefore, naïve and TCM cells were distinguished based on scatter properties using backgating on the TNFα+ population to tighten the TCM gate.

### DETAILED DESCRIPTION

The invention is based at least in part on the discovery of methods for isolating viable T cell populations. One method includes contacting T cells with an activator to obtain activated T cells and also contacting the T cells with an agent that binds to a cell surface marker during at least part of the time during which the T cell is contacted with the activator. An exemplary method comprises (i) contacting a population of cells comprising a T cell with a first activator that binds to a T cell receptor (TCR) on the T cell thereby activating the T cell and (ii) a first agent that binds to a first cell surface molecule of the T cell, to obtain a T cell population bound by the first agent; and (ii) isolating the T cell population by a method using the first agent. The methods allow isolation of viable, or live T cells.

Definitions:

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “activation marker” includes several cytokines (IL-2 and others), IL-2 receptor subunit a (CD25), insulin receptor, transferrin receptor and several other surface molecules such as CTLA-4, CD40L, CD 26, CD30, CD54, CD69 and CD70 that are expressed following antigen stimulation of T cells.

An “activator” of a T cell refers to a stimulus that activates T cells and include antigens, which may be presented on antigen presenting cells or on other surfaces; polyclonal activators, which bind to many T cell receptor (TCR) complexes regardless of specificity, and include lectins, e.g., concanavalin-A (Con-A) and phytohemagglutinin (PHA) and agents such as antibodies that bind specifically to invariant framework epitopes on TCR or CD3 proteins; and superantigens, which stimulate a significant number of T cells, and include, e.g., enterotoxins, such as Staphyloccal enterotoxins.

The term “antigen-presenting matrix” refers to a molecule or molecules, e.g., a surface, that can present antigen in such a way that the antigen can be bound by a T cell antigen receptor on the surface of a T cell. An antigen-presenting matrix can be part of an antigen-presenting cell (APC), a vesicle preparation of an APC, or can be in the form of a synthetic matrix on a bead or a plate. The term “antigen presenting cell”, as used herein, refers to any cell that presents on its surface an antigen in association with a MHC or portion thereof, or, one or more non-classical MHC molecules, or a portion thereof.

The term “antibody” refers to immunoglobulin molecules and antigen-binding portions of immunoglobulin molecules, i.e., molecules that contain an antigen binding site which specifically binds (“immunoreacts with”) an antigen. In an exemplary embodiment, the term “antibody” specifically covers monoclonal antibodies (including agonist, antagonist, and blocking or neutralizing antibodies). Structurally, the simplest naturally occurring antibody (e.g., IgG) comprises four polypeptide chains, two heavy (H) chains and two light (L) chains inter-connected by disulfide bonds.

As used herein “antigen stimulation” of T cells is achieved by exposing the cells to at least one antigen under conditions effective to elicit antigen-specific stimulation of at least one T cell.

“CD4+ T cells” refers to a subset of T cells that express CD4 on their surface and are associated with cell-mediated immune response. They are characterized by the secretion profiles following stimulation, which may include secrection of cytokines such as IFN-gamma, TNF-alpha, IL-2, IL-4 and IL-10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

“CD40L” refers to a ligand for CD40, a receptor that is a member of the TNF receptor super family. CD40L is expressed on activated T cells. CD40L is responsible for transducing signal via CD40, which is known to be expressed, for example, by B lymphocytes. Full-length CD40-L is a membrane-bound polypeptide with an extracellular region at its C terminus, a transmembrane region, and an intracellular region at its N-terminus. Other terms commonly used to describe CD40L are T-BAM, or gp39.

“CTLA-4” refers to the cytolytic (cytotoxic) T-lymphocyte-associated antigen number 4 receptor (“CTLA-4 receptor”; also referred to herein as “CTLA-4”). CTLA-4 is a protein that is expressed on the surface of T-cells and binds to the protein ligands B7-1 and B7-2 (Linsley et al., Immunity, 1:793, 1994; Linsley et al., J. Exp. Med., 173: 721, 1991). B7-1 and B7-2 are expressed on the surface of immune system cells known as antigen presenting cells (“APCs”).

An “effective amount” is an amount sufficient to effect a beneficial or desired clinical result upon treatment. An effective amount can be administered to a patient in one or more doses. In terms of treatment, an effective amount is an amount that is sufficient to palliate, ameliorate, stabilize, prevent, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition and the form and effective concentration of the antigen-binding fragment administered.

The term “substantially enriched” cell population, refers to a cell population is at least about 50-fold, more preferably at least about 500-fold, and even more preferably at least about 5000-fold or more enriched from an original mixed cell population comprising the desired cell population.

The terms “T lymphocyte” and “T cell” are used interchangeably, and refer to a cell that displays on its surface one or more antigens characteristic of T cells, such as, for example, CD2 and CD3. A T cell is a cell that expresses a T cell antigen receptor (TCR) capable of recognizing antigen when displayed on the surface of antigen presenting cells or matrix together with one or more MHC molecules or, one or more non-classical MHC molecules.

As used herein, “treatment” is an approach for obtaining beneficial or desired clinical results. For purposes of this invention, beneficial or desired clinical results include alleviation of symptoms, diminishment of extent of disease, stabilized (i.e., not worsening) state of disease, preventing spread (i.e., metastasis) of disease, delay or slowing of disease progression, amelioration or palliation of the disease state, and remission (whether partial or total). “Treatment” can also mean prolonging survival as compared to expected survival if not receiving treatment.

Sources of Populations of Cells

Populations of cells for use in the methods described herein may be mammalian cells, such as a human cells, non-human primate cells, rodent cells (e.g., mouse or rat), bovine cells, ovine cells, porcine cells, equine cells, sheep cell, canine cells, and feline cells or a mixture thereof. Non-human primate cells include rhesus macaque cells. The cells may be obtained from an animal, e.g., a human patient, or they may be from cell lines. If the cells are obtained from an animal, they may be used as such, e.g., as unseparated cells (i.e., a mixed population); they may have been established in culture first, e.g., by transformation; or they may have been subjected to preliminary purification methods. For example, a cell population may be manipulated by positive or negative selection based on expression of cell surface markers; stimulated with one or more antigens in vitro or in vivo; treated with one or more biological modifiers in vitro or in vivo; or a combination of any or all of these. In an illustrative embodiment, a cell population is subjected to negative selection for depletion of non-T cells and/or particular T cell subsets. Negative selection can be performed on the basis of cell surface expression of a variety of molecules, including B cell markers such as CD19, and CD20; monocyte marker CD14; the NK cell marker CD56.

Populations of cells include peripheral blood mononuclear cells (PBMC), whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include immunized donors, non-immunized (naive) donors, treated or untreated donors. A “treated” donor is one that has been exposed to one or more biological modifiers. An “untreated” donor has not been exposed to one or more biological modifiers.

Methods of obtaining populations of cells comprising a T cell are well known in the art. For example, PBMC can be obtained as described according to methods known in the art. Examples of such methods are set forth in the Examples and is discussed by Kim, C. H. et al. (J. Virol. 66:3879-3882 (1992)); Biswas, B. et al. (Annals NY Acad. Sci. 590:582-583 (1990)); Biswas, B. et al. (J. Clin. Microbiol. 29:2228-2233 (1991)).

It is also possible to obtain a cell sample from a subject, and then to enrich it for a desired cell type. For example, PBMCs can be isolated from blood as described herein. Counter-flow centrifugation (elutriation) can be used to enrich for T cells from PBMCs. Cells can also be isolated from other cells using a variety of techniques, such as isolation with an antibody binding to an epitope on the cell surface of the desired cell type. Another method that can be used includes negative selection using antibodies to cell surface markers to selectively enrich for a specific cell type without activating the cell by receptor engagement.

Bone marrow cells may be obtained from iliac crest, femora, tibiae, spine, rib or other medullary spaces. Bone marrow may be taken out of the patient and isolated through various separations and washing procedures. A known procedure for isolation of bone marrow cells comprises the following steps: a) centrifugal separation of bone marrow suspension in three fractions and collecting the intermediate fraction, or buffycoat; b) the buffycoat fraction from step (a) is centrifuged one more time in a separation fluid, commonly Ficoll (a trademark of Pharmacia Fine Chemicals AB), and an intermediate fraction which contains the bone marrow cells is collected; and c) washing of the collected fraction from step (b) for recovery of re-transfusable bone marrow cells.

If one desires to use a population of cells enriched in T cells, such populations of cells can be obtained from a mixed population of cells by leukapheresis and mechanical apheresis using a continuous flow cell separator. For example, T cells can be isolated from the buffy coat by any known method, including separation over Ficoll-Hypaque™ gradient, separation over a Percoll gradient, or elutriation.

Activation of T Cells

In one embodiment, a population of cells comprising a T cell is contacted with a first activator that binds to a T cell receptor on the T cell, thereby activating the T cell. Activated T cells are characterized by, inter alia, transcriptional activation of a variety of genes; expression of new cell surface molecules; secretion of effector cytokines and/or performance of cytolytic functions; and induction of mitotic activity. Activators, which activate T cells, include molecules that bind to the antigen-binding region of T cell receptors, such as antigens. Antigens include peptides; proteins; glycoproteins; lipids; glycolipids; all of which may be present on cells, in cell extracts, tissue extracts, whole microorganisms such as protozoans, bacteria, and viruses. Antigens can be unmodified, i.e., used in their native state. Alternatively, an antigen can be modified by any known means, including heating, for example to denature a protein or to inactivate a pathogen; chemical modification to denature a protein, or to cross-link two antigen molecules; glycosylation; chemical modification with moieties including polyethylene glycol; and enzymatic digestion.

Generally, an antigen can be any molecule that one desires to have T cells specific to. Antigens can be, e.g., associated with a condition to be treated, and may be molecules present on cancer cells; pathogenic organisms such as bacteria and viruses; and auto-immune cells. The antigen can be a single antigen with a single antigenic determinant; a single antigen with multiple antigenic determinants or a mixture of antigens. Accordingly, the antigen can be an autoantigen or a foreign antigen, depending on the condition to be treated. Autoantigens include antigens associated with autoimmune diseases and those associated with cancer cells. If the antigen is an autoantigen, the autoantigen can be part of an organ, for example the brain or the thyroid gland and need not be purified therefrom. However, purified autoantigens or mixtures of purified autoantigens can also be used. Antigens that may be used are further described herein.

In one embodiment, activation of a T cell with an antigen is conducted by presenting the antigen in combination with an antigen-presenting matrice. In a particular embodiment, an antigen presenting matrice is the surface of an antigen-presenting cell (APC) comprising an antigen presenting molecule. The antigen-presenting molecule can be a major histocompatibility complex (MHC) molecule, which can be class I or class II or, a non-classical MHC molecule such as CD1; an MHC epitope; a fusion protein comprising an MHC epitope; or a synthetic MHC epitope. The nature of the antigen-presenting molecule is not critical, so long as it is capable of presenting antigen to a T cell. Methods of preparing MHC epitopes are known in the art. For example, APCs suitable for use in the present invention are capable of presenting an antigen to T cells in association with an antigen-presenting molecule, such as an MHC molecule. APCs include macrophages, dendritic cells, CD40-activated B cells, antigen-specific B cells, tumor cells, virus-infected cells and genetically modified cells.

APCs can be obtained from a variety of sources, including peripheral blood mononuclear cells (PBMC), whole blood or fractions thereof containing mixed populations, spleen cells, bone marrow cells, cells obtained by leukapheresis, and lymph nodes, e.g., lymph nodes draining from a tumor. APCs can be treated in vitro with one or more biological modifiers, cytokines such as IL-2, IL-4, IL-10, TNF-alpha, IL-12, IFN-gamma; non-specific modifiers such as phytohemagglutinin (PHA), phorbol esters such as phorbol myristate acetate (PMA), concanavalin-A, and ionomycin; antibodies specific for cell surface markers, such as anti-CD2, anti-CD3, anti-IL-2 receptor, anti-CD28; chemokines, including, for example, lymphotactin. The biological modifiers can be native factors obtained from natural sources, factors produced by recombinant DNA technology, chemically synthesized polypeptides or other molecules, or any derivative thereof having the functional activity of the native factor.

APCs are generally alive but can also be irradiated, mitomycin C treated, attenuated, or chemically fixed. Further, the APCs need not be whole cells. Instead, vesicle preparations of APCs can be used.

Cells which do not normally function in vivo in mammals as APCs can be modified to function as APCs. A wide variety of cells can function as APCs when appropriately modified. Examples of such cells are insect cells, for example Drosophila or Spodoptera; foster cells, such as the hunan cell line T2, which bears a mutation in its antigen presenting pathway that restricts the association of endogenous peptides with cell surface MHC class I molecules. Zweerink et al. (1993) J. Immunol. 150:1763-1771. For example, expression vectors which direct the synthesis of one or more antigen-presenting polypeptides, such as MHC molecules, and, optionally, accessory molecules can be introduced into these cells to effect the expression on the surface of these cells antigen presentation molecules and, optionally, accessory molecules or functional portions thereof. Accessory molecules include co-stimulatory antibodies such as antibodies specific for CD28, CD80, or CD86; costimulatory molecules, including B7.1 and B7.2; adhesion molecules such as ICAM-1 and LFA-3; and survival molecules such as Fas ligand and CD70. See, for example, PCT Publication No. WO 97/46256. Alternatively, antigen-presenting polypeptides and accessory molecules which can insert themselves into the cell membrane can be used. For example, glycosyl-phosphotidylinositol (GPI)-modified polypeptides can insert themselves into the membranes of cells. Medof et al. J. Exp. Med. 160:1558-1578; and Huang et al. Immunity 1:607-613.

A person of skill in the art will recognize that naturally occurring APCs can also be genetically modified, such as genetically modified, by, e.g., increasing the number of antigen presenting molecules, accessory molecules or other. For example, expression of a polynucleotide encoding an MHC molecule under transcriptional control of a strong promoter such as the CMV promoter, can result in high level expression of the MHC molecule on the cell surface, thus increasing the density of antigen presentation. Alternatively, an APC can be transfected with a polynucleotide construct comprising a polynucleotide encoding an antigen such that the antigen is expressed on the cell surface together with an MHC molecule. Antigens can also be introduced into APCs, such as by contacting APCs with antigens.

Genetic modifications can be introduced according to methods known in the art. In particular, a nucleotide sequence encoding a polypeptide of interest is preferably operably linked to control sequences for transcription and translation. A control sequence is “operably linked” to a coding sequence if the control sequence regulates transcription or translation. Any method in the art can be used for the transformation, or insertion, of an exogenous polynucleotide into an APC, f6r example, lipofection, transduction, infection or electroporation, using either purified DNA, viral vectors, or DNA or RNA viruses. The exogenous polynucleotide can be maintained as a non-integrated vector, for example, a plasmid, or, can be integrated into the host cell genome.

Alternatively, a synthetic antigen-presenting matrix can be used to present antigen to T cells. A synthetic matrix can be a solid support, for example, beads or plates, which include an antigen presenting molecule, preferably an MHC Class I or MHC Class II molecule. A synthetic matrix may further comprise one or more accessory molecules, adhesion molecules such as ICAM-1 and LFA-3; and survival molecules such as Fas ligand and CD70. Portions of these molecules can also be used, as long as their function is maintained. Solid supports include metals or plastics, porous materials, microbeads, microtiter plates, red blood cells, and liposomes. See, for example, PCT Publication No. WO 97/46256; and WO 97/35035.

In another embodiment, an activator is a molecule that does not bind to the antigen-binding region of T cell receptors. For example, an activator can be a polyclonal activator, such as lectins, e.g., concanavalin-A (Con-A) and phytohemagglutinin (PHA) and agents such as antibodies that bind specifically to invariant framework epitopes on TCR or CD3 proteins. Other activators include superantigens, such as enterotoxins, e.g., Staphyloccal enterotoxins. Polyclonal activators and superantigens are commercially available. A population of cells may be contacted with two or more activators, either simultaneously or sequentially. In a particular embodiment, a population of cells comprising a T cell is contacted with two or more antigens present on APCs, to thereby activate T cells having T cell receptors binding specifically to two or more antigens. Such populations of T cells would be polyclonal.

A population of cells comprising at least one T cell may be contacted with an activator for a period of time sufficient for the T cell to be activated. In a preferred embodiment, the time frame is sufficient for the cell surface molecule to appear on the cell surface (see below). For example, a population of cells may be contacted with an activator for at least about 10 minutes; 1 hour; 3 hours; 5 hours; 7 hours; 10 hours; 12 hours; 15 hours; 24 hours; 2 days; 3 days; 5 days or 7 days.

Methods for determining whether an activator is capable of activating T cells or for determining the length of time necessary for T cell activation are known in the art and include, for example, ³H-thymidine uptake by effector cells, cytokine production by effector cells, and cytolytic ⁵¹Cr-release assays.

Isolation of T Cells

In one embodiment, a T cell that has been activated, e.g., as described above, is isolated by contacting the T cell with an agent that binds to a cell surface molecule on the T cell, and separating the T cell from the other cells by a method using the agent.

In a preferred embodiment, the cell surface molecule is a molecule that is present on activated T cells. Examples include “cluster of differentiation” cell surface markers such as CD2, CD3, CD4, CD8, TCR, CD45, CD45RO, CD45RA, CD11b, CD26, CD27, CD28, CD29, CD30, CD31, CD40L, CTLA-4; lymphocyte activation gene 3 product (LAG3); signaling lymphocyte activation molecule (SLAM); T1/ST2; chemokine receptors such as CCR3, CCR4, CXCR3, CCR5; homing receptors such as CD62L, CD44, CLA, CD146, alpha 4 beta 7, alpha E beta 7; activation markers such as CD25, CD69 and OX40; and lipoglycans presented by CD1.

The surface molecule may be a molecule that is present only in activated T cells, as opposed to resting T cells. Exemplary cell surface molecules are activation markers, such as CD40L, CTLA-4, CD69, CD25, the transferrin receptor, insulin receptor and VLA-4.

The choice of cell surface molecule may depend on the type of T cells one desires to isolate. Since subpopulations of T cells, e.g., CD4+ and CD8+ T cells, have different cell surface molecules, using agents that bind to these specific cell surface molecules will allow isolation of T cells from specific subpopulations of T cells. For example, CD4+ T cells may be isolated using agents that bind to CD40L and CTLA-4. CD8+ T cells may be isolated using agent that bind to CTLA-4, the transferrin receptor, insulin receptor and VLA-4.

An agent that binds to a cell surface molecule can be any type of molecule provided that it binds to the cell surface molecule with sufficient affinity to allow isolation of a significant portion of T cells having the cell surface marker by a method using the agent. Accordingly, the type of the agent will vary depending on the method of isolation used. However, generally, the affinity of binding (Km) of an agent to a cell surface molecule will be at least about 10⁻⁶ M, 10⁻⁷ M, 10⁻⁸ M, 10⁻⁹ M, 10⁻¹⁰ M, 10⁻¹¹ M, or 10⁻¹² M.

The agent can be an antibody or portion thereof sufficient for binding specifically to an antigen. An antibody, or generally any molecule, “binds specifically” to an antigen (or other molecule) if the antibody binds preferentially to the antigen, and, e.g., has less than about 30%, preferably 20%, 10%, or 1% cross-reactivity with another molecule. Portions of antibodies include Fv and Fv′ portions. Antibodies can be naturally-occurring antibodies, e.g., monoclonal antibodies obtained by the method of Koehler and Milstein and polyclonal antibodies obtained, e.g., by injection of an antigen into an animal. Antibodies can also be partially or fully humanized antibodies, single chain antibodies or other variants of antibodies. An agent can also be a ligand or receptor or portion thereof of a receptor or ligand, respectively. For example, an agent for binding to CD40L can be CD40 or a portion thereof.

Agents binding to cell surface markers can be obtained commercially. Examples of commercially available antibodies binding to human activation markers include phycoerythrin or FITC conjugated mouse anti-human CD152 antibody (CTLA-4) (Chemicon); phycoerythrin labeled mouse anti-human CD152 antibody (CTLA-4) (Beckman Coulter); phycoerythrin labeled mouse anti-human CD152 antibody (CTLA-4) (Bioscience); biotin or FITC labeled anti-human CD40L antibody (Caltag); FITC or phycoerythrin labeled mouse anti-human CD154 antibody (CD40L) (Chemicon); and FITC labeled anti-human CD154 (CD40L) antibody (eBioscience). Anti-CD69 antibodies can be obtained from the following sources: FITC, phycoerythrin, Cy-chrome, allophycocyanin or R-phycoerythrin-Cyanine 7 labeled mouse anti-human CD69 antibody (BD Biosciences Pharmingen); FITC or phycoerythrin labeled mouse anti-rhesus CD69 antibody (BD Biosciences Pharmingen); phycoerythrin labeled mouse anti-human CD69 antibody (Beckman Coulter); FITC, phycoerythrin, allophycocyanin, phycoerytirin-Cyanine 5, or biotin labeled mouse anti-human CD69 antibody (CalTag); and FITC or phycoerythrin labeled anti-human CD69 antibody (eBioscience). Alternatively, agents can also be prepared according to methods well known in the art, e.g., in the art of antibody making.

Depending on the method of isolation used (see below), agents may comprise a label, e.g., a fluorescent or magnetic label. In such embodiments, the agent is said to be “directly labeled.” An agent can also be “indirectly labeled,” i.e., the label is attached to the agent through one or more other molecules, e.g., biotin-streptavidin. Alternatively, the agent is not labeled, but is later contacted with a binding agent after the agent is bound to a T cell. For example, the agent may be an antibody, referred to as a “primary antibody” and the binding agent is a second antibody or “secondary antibody” that binds to the Fc portion of the first antibody. Labels may be linked, preferably covalently, to agents according to methods known in the art.

Further depending on the method of isolation used, agents may be linked to a solid surface, e.g., beads and plates. Methods for direct chemical coupling of agents, e.g., antibodies, to the cell surface are known in the art, and may include, for example, coupling using glutaraldehyde or maleimide activated antibodies. Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Biotinylation can be accomplished by, for example, treating the cells with dithiothreitol followed by the addition of biotin maleimide.

Agents are preferably contacted with the population of cells comprising T cells at least for a time sufficient for the agent to bind to a cell surface molecule on the T cell. For example, an agent may be contacted with a population of cells for at least about 10 minutes, minutes, 1 hour, 3 hours, 5 hours, 7 hours, 10 hours, 15 hours, 1 day, 3 days, 7 days or days. The agent is preferably contacted with the population of cells for at least some time during T cell activation, i.e., when the population of cells is contacted with an activator. In an illustrative embodiment, a population of cells comprising at least one T cell is contacted simultaneously with an activator and with an agent for at least 10 minutes. The agent may be added to the population of cells before the activator is added, or after the activator is added. The agent may further be contacted with the cells after the T cells have been activated, e.g., after the activator has been removed from the cells.

Agents may be added to populations of cells comprising T cells that are, e.g., at a concentration of about 0.5 to 5.0×106 cells per ml. The particular concentration of agent used will depend on the type of agent and the surface molecule and can be determined, e.g., according to methods known in the art.

Methods of Isolating T Cells

Analysis of the cell population and cell sorting based upon the presence of an agent can be accomplished by a number of techniques known in the art. Cells can be analyzed or sorted by, for example, flow cytometry or fluorescent activated cell sorting (FACS). These techniques allow the analysis and sorting according to one or more parameters of the cells, such as the presence of cell surface molecules, cell size and DNA content. For example, dead cells can be eliminated by selection with dyes associated with dead cells e.g., (propidium iodide, LDS). Red blood cells can be removed by (for example) elutriation, hemolysis, or Ficoll-Paque gradients. These methods are further described herein and in, for example, The Handbook of Experimental Immunology, Volumes 1 to 4, (D. N. Weir, editor); Flow Cytometry Cell Sorting (A. Radbruch, editor, Springer Verlag, 1992); and Cell Separation Methods and Applications (D. Recktenwald and A. Radbruch, eds., 1997) Marcel Dekker, Inc. N.Y.

Other methods for cell sorting include, for example, palming and separation using affinity techniques, including those techniques using solid supports such as plates, beads and columns.

In particular, cell sorting may utilize magnetic separations, which may use magnetic beads. Magnetic beads are available from a number of sources, including for example, Dynal (Norway), Advanced Magnetics (Cambridge, Mass., U.S.A.), Immuncon (Philadelphia, U.S.A.), Immunotec (Marseilles, France), and Miltenyi Biotec GmbH (Germany).

Magnetic labeling methods may include colloidal superparamagnetic particles in a size range of 5 to 200 nm, preferably in a size of 10 to 100 nm. These magnetic particles allow quantitative magnetic labeling of cell and are available, for example, through Miltenyi Biotec GmbH.

Immunospecific fluorescent or magnetic liposomes can also be used for isolating T cells. In these cases, the liposomes contain magnetic material and/or fluorescent dyes conjugated with antibody or other agent on their surfaces, and magnetic or fluorescent based separation is used to separate T cells having a cell surface molecule that is recognized by the agent, and cells that do not have such surface molecules. strengths of the two opposed forces. Typical opposed forces are, for example, forces induced by magnetic fluids mixed in the separation medium in the magnetic separation chamber, gravity, and viscous forces induced by flow speed of medium relative to the cell.

High gradient magnetic separation (HGMS), a procedure for selectively retaining magnetic materials in a chamber or column disposed in a magnetic field, may be used. In one application of this technique T cells are attached to magnetic particles. The attachment is generally through association of the cell with an agent that is directly or indirectly conjugated to the coating on a magnetic particle. Cells thus coupled to a magnetic label, are suspended in a fluid which is then applied to a chamber. In the presence of a magnetic gradient supplied across the chamber, the magnetically labeled cells are retained in the chamber, whereas cells that do not have or have only a low amount of magnetic label pass through the chamber. A chamber may also contain a matrix, in which case, labeled cells may become associated with the matrix.

Retained cells can then be eluted by changing the strength of, or by eliminating, the magnetic field or by introducing a magnetic fluid. The chamber across which the magnetic field is applied may be provided with a matrix of a material of suitable magnetic susceptibility to induce a high magnetic field gradient locally in the chamber in volumes close to the surface of the matrix. This permits the retention of fairly weakly magnetized particles. Publications describing a variety of HGMS systems include U.S. Pat. Nos. 4,452,773, 4,230,685, PCT application W085/04330, U.S. Pat. No. 4,770,183, and PCT/EP89/01602; and U.S. Pat. Nos. 5,411,863; 5,543,289; 5,385,707; and 5,693,539.

In certain embodiments, a T cell population isolated as described herein is further enriched in particular T cells, e.g., by methods comprising (i) contacting the T cell population with (a) a second activator that binds to the T cell receptor on at least some cells of the T cell population thereby activating at least some cells of the T cell population and (b) a second agent that binds to a second cell surface molecule of at least some cells of the T cell population, to obtain a T cell population bound by the second agent; and (ii) isolating the T cell population by a method using the second agent. The second activator may be the same or different from the first activator. For example, for further enrichment of a population of cells in particular antigen-specific T cells, one may contact a second (or third or more) time with the same antigen. Alternatively, one can contact the cell population with a different antigen to, e.g., eliminate any T cells that are reactive against the latter antigen. One can also use an antigen as a first activator and a polyclonal activator or superantigen as a second activator or vice versa.

In the second round of selection, the second agent may be the same or different from the first agent that may bind to the same or different cell surface molecule. For example, the first cell surface molecule may be CD40L and the second cell surface molecule may be CTLA-4. In other embodiments, the first and the second agent are different antibodies that bind to the same cell surface molecule. This latter combination of agents may be useful to eliminate any cells that were isolated during the first round due to some cross-reactivity of the antibody.

It is also contemplated that different separation methods can be combined, for example, magnetic cell sorting can be combined with FACS. For example, a first round of purification, i.e., with a first activator and a first agent can be performed with a first method and the second round of purification performed with a second method.

A person of skill in the art will recognize that several more round of purification can be conducted to obtain a population of cells of the desired composition.

To enhance or sustain activity and/or proliferation of isolated T cells, the cells may be cultured and restimulated, e.g., with antigen of interest or a polyclonal activator, -after many days in culture, for example, after 5 or 10 days in culture. The incubation is preferably also in the presence of lymphokine(s), e.g., purified IL-2 and/or concanavalin A (con A)—stimulated spleen cell supernatant or conditioned medium from activated T cell culture. Preferably, human IL-2 is used with human cells. By repeated stimulations for example, carried out every 3 to 9 days, a continuous antigen-specific T cell culture or T cell line with specificity for the antigens present on the antigenic cells can be maintained or established. Standard methods of T cell cloning and clonal expansion may be applied to further propagate these T cells. See generally, Fathman et al., 1989, in Chapter 30, in “Fundamental Immunology” 2nd edition, ed. Paul, W. E., Raven Press, New York, pp 803-815. Cells may be tested for reactivity on day six after restimulation.

Isolated Viable Antigen-specific or Polyclonal T Cell Populations

Provided herein are compositions comprising T cells. For example, methods described herein allow the isolation of viable cell populations, wherein at least about 50%, 70%, 80%, 90%, 95%, 98% or 99% of the cell population consists of viable T cells. The cell populations may be antigen-specific or polyclonal, depending on the type of activator, e.g., whether an antigen or a polyclonal activator, that was used. Isolated populations of T cells may also comprise T cells having T cell receptors specific for two or more antigens. The percentage of T cells that are antigen-specific can be readily determined, for example, by a ³H-thymidine uptake assay in which the T cell population is challenged by an antigen-presenting matrix presenting the desired antigen(s).

The T cells in an isolated population of cells may be of particular T cell subpopulations. For example, at least 90% of an isolated viable cell population may consist of CD4+ T cells, CD8+ T cells, CD40L+, CTLA4+, CD69+ or CD25+ T cells, or any combination thereof. A population of cells may also comprise a certain proportion of T cells characterized by a particular secretion profile when activated. For example, a population of cells may comprise a certain percentage of IFN-gamma or TNF-alpha secreting T cells. They may also comprise a certain number of Th or Tc cells. Cell compositions may also comprise feeder cells, or other components necessary for maintaining T cells.

Since the methods described herein allow the enrichment of a population of cells in T cells or particular subpopulations thereof, provided herein are enriched cell populations. By “enriched” is meant that a cell population is at least about 10 fold, 50-fold, more preferably at least about 500-fold, and even more preferably at least about 5000-fold or more enriched from an original mixed cell population.

Cells may be frozen or in solution. Cells may be in a container, e.g., a means for administrating the cells to a subject, such as a syringe or attached to a stent.

Cells may also be further modified, e.g., by genetic manipulation. For example, nucleic acids encoding particular proteins may be introduced into the populations of T cells. Proteins to be expressed in the cells include those that may provide a medical benefit, such as a cytokine or growth factor or an immunomodulatory protein. Alternatively, factors that affect the biology of T cells can be introduced. For example, genes encoding a different T cell receptor or proteins associated therewith may be introduced. The genes that are introduced into the cells may be under the control of an inducible promoter. Methods for introducing and expressing exogenous genes or inhibiting the expression of endogenous genes by introduction of particular genetic constructs in cells may be accomplished with viral-based or non-viral based vectors. Viral vectors include adenoviral vectors, adenovirus associated viral (AAV) vectors and lentivirus based vectors.

Therapeutic Methods

Provided herein are method for treating a first subject, comprising (i) obtaining a population of cells comprising a T cell from the subject; (ii) subjecting the population of cells to a method described herein to thereby obtain a T cell population; and (iii) administering the T cell population to a second subject. The first and the second subject may the same or different. If the subjects are the same, the procedure is an autologous procedure and the cells are autogeneic. If a subject receives cells originally isolated from a different individual, the cells may be allogeneic cells. T cell populations may be purified by one or more rounds of purification and may be expanded before, after or between rounds of purification.

When cells are allogeneic, the cells are preferably depleted of alloreactive cells before use. This can be accomplished by any known means, including, for example, mixing the allogeneic T cells and a recipient cell population and incubating them for a suitable time, then depleting CD69+ cells, or inactivating alloreactive cells, or inducing anergy in the alloreactive cell population. Methods described herein can also be used for this purpose.

In one embodiment, populations of T cells described herein, e.g., populations of CD4+ or CD8+ T cells, may be used for treating cancer. Examples of antigen-specific T cells selected for the treatment of cancer may include IFN-gamma or TNF-alpha secreting CD8+ T cells (cytotoxic T cells).

Populations of cells comprising at least one T cell may be isolated from, e.g., the blood, bone marrow or a tumor of a subject, containing tumor infiltrating lymphocytes. Tumor infiltrating lymphocytes are further described in, e.g., U.S. Pat. No. 5,126,132. These cells are then subjected to the methods described herein to obtain enriched populations of T cells. The cell surface molecule that will be targeted may be one allowing isolation of CD4+ or CD8+ cells. The activator to use may be antigen presenting cells obtained from the same subject, and may be, e.g., peripheral blood mononuclear cells (PBMCs) or tumor cells or immune cells located close to or in the tumor cells. By way of example, antigen presenting cells can be obtained from a tumor that is surgically removed. Prior to use, the solid cancer tissue or aggregated cancer cells should preferably be dispersed, e.g., mechanically, into a single cell suspension by standard techniques. Enzymes such as collagenase and Dnase may also be used to disperse cancer cells. Cell lines, in particular cell lines expressing cancer associated antigens, may also be used.

In another embodiment, the antigen used to stimulate T cells is a cancer associated antigen, and the antigen is incubated with antigen presenting cells obtained, e.g., form the subject to be treated. Such antigens may include Her2, p53, VEGF, ras, myc, mdm2. The antigen chosen will depend on the disease to be treated.

Populations of T cells reactive against human cancer cells can be used, alone or in conjunction with surgery, chemotherapy, radiation or other anti-cancer therapies, to eradicate metastases or micrometastases. For example, to eradicate or to inhibit the growth of metastases or micrometastases, populations of T cells can be administered to a subject having or suspected of having metastases or micrometastases.

Cancers that can be treated or prevented include sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

Populations of T cells reactive against cancer cells can also be used to purge bone marrow of cancer cells prior to bone marrow transplantation. For example, bone marrow from a donor can be contacted in vitro with the T cells, so that the T cells lyse any residual cancer cells in the bone marrow, prior to administering the bone marrow to the subject, e.g., for purposes of hematopoietic reconstitution. T cells for this purpose may have been obtained using as an activator cancer cells or APCs presenting cancer antigens.

In another embodiment, infections with pathogenic organisms can be treated or prevented. For example, a population of cells comprising at least one T cell can be obtained from a subject to be treated, subjected to the methods described herein, wherein the activator is an antigen from the pathogen or antigen presenting cells presenting the antigen and obtained from the subject; and reinfused into the subject. CD4+ as well as CD8+ cells can be reinfused.

Infectious diseases may be caused by infectious agents including viruses, bacteria, fungi, protozoans and parasites. Viral diseases that can be treated or prevented by the methods described herein include those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsachie virus, mumps virus, measles virus, rubella virus, polio virus, human immunodeficiency virus type I (HIV-I), and human immunodeficiency virus type II (HIV-II). Bacterial diseases that may be treated or prevented as described herein include those caused by bacteria including mycobacteria rickettsia, mycoplasma, neisseria and legionella. Protozoal diseases that may be treated or prevented as described herein include those caused by protozoa including leishmania, kokzidioa, and trypanosoma. Parasitic diseases that may be treated or prevented as described herein include those caused by parasites including chlamydia and rickettsia.

For example, CD4⁺ T helper cells play an important role in maintaining effective immunity against viral pathogens (Kalams et al. 1998). In murine models, virus-specific T helper responses are important in maintaining effective CTL responses against viral pathogens such as MHV-68, a murine lymphotropic herpesvirus (Cardin et al. 1996), and lymphocytic choriomeningitis virus (Matloubian et al. 1994). In the absence of CD4⁺ T cell responses, virus-specific CTL may either be eliminated or persist yet be nonfunctional (Zajac et al. 1998). In HIV and SIV infection, virus-specific T helper responses as assessed by standard proliferation assays are generally low to absent (Wahren et al. 1987; Dittmer et al. 1994). However, recent studies have demonstrated that vigorous HIV-1 specific CD4⁺ T lymphocyte proliferative responses can be detected in HIV-1-infected long term nonprogressors and in subjects treated with antiretroviral therapy early in the course of HIV infection (Rosenberg et al. 1997). Virus-specific CD4+ T cells also appear to play an important role in the maintenance of CD8+ T cell responses against cytomegalovirus in humans (Walter et al. 1995). Taken together, these pieces of evidence support a significant role for antigen-specific CD4+ T cells in immune responses to viral pathogens and also highlight the clinical utility of these cells for adoptive transfer.

Another use of enriched T cell populations of the present invention is in immunomodulation, for example, in the treatment of autoimmune disorders, inflammatory disorders, allergies and hypersensitivities such as delayed-type hypersensitivity and contact hypersensitivity. T cells which are capable of destroying or suppressing the activity of autoreactive cells can be enriched in vitro, optionally expanded in vitro, then re-introduced into a patient. In the treatment of allergic responses, the ratio of TH1 to TH2 cells can be altered, or, cells reactive toward allergen-specific cells can be enriched and introduced into an individual. Inducing T cell anergy can also be used to treat, ameliorate or prevent allograft rejection thus improving the results of organ transplantation and increasing the range of histotypes to which a patient can be made histocompatible. Examples of T cells that may be used in the treatment for suppression and/or counter-regulation of allergy or vaccination against allergy diseases may include IL-10 or TGF-beta secreting CD4+ T cells and IL-4 secreting CD4+ T cells.

The compositions of cells can be administered by any known route, including intravenously, parenterally, or locally. In the treatment methods of the present invention, enriched antigen-specific T cells are administered to an individual. The total number of cells, the number of doses, and the number of cells per dose will depend upon the condition being treated. Generally, about 10⁶ to 10¹¹ cells are administered in a volume ranging from about 5 ml to 1 liter. The cells can be administered in a single dose or in several doses over selected time intervals. Of the cells being administered, preferably at least about 10%, more preferably at least about 20%, more preferably at least about 50%, are T cells of a desired subpopulation and reactivity.

Compositions comprising enriched antigen-specific T cell populations can further be used as vaccines, to prevent or substantially reduce the probability of the occurrence of a disease state such as a viral infection, autoimmune disorder, allergic response, cancer, or other disorder, or will reduce the severity or duration of the disease if subsequently infected or afflicted with the disease.

Diagnostic Methods

The methods described herein may also be used for diagnostic purposes. For example, particular populations of T cells may be isolated from a subject and used for determining a particular characteristic of T cells in that population. In an illustrative embodiment, a population of viable T cells is isolated and the reactivity of the T cells towards self antigens is determined, so as to determine whether a subject has an autoimmune disease, and optionally to identify the particular antigen to which the subject is reacting. The method may involve obtaining blood cells from a subject having an autoimmune disease, isolating viable T cells as described herein, and testing the T cells for reactivity against self antigens. Similar diagnostic methods may also be used for other diseases, in which one desires obtaining characteristics of T cells or subpopulations of T cells.

Other Uses

Isolated viable T cells may also be used in vitro for isolating particular cell products from the cells. In particular, pure populations of T cells provide the advantage of being able to isolate more efficiently products of those particular T cells. The T cells can also be genetically modified and used to isolate a recombinant product.

Kits

Also provided are kits. The kits may contain materials for therapeutic or diagnostic purposes or materials for obtaining populations of T cells as described herein. For example, a kit may comprise one or more activators and one or more agents that bind to T cell surface molecules. Buffers for use in the method and other reagents, e.g., labels, may also be provided.

All publications and patents mentioned herein are hereby incorporated by reference in their entirety as if each individual publication or patent was specifically and individually indicated to be incorporated by reference. In case of conflict, the present application, including any definitions herein, will control.

The practice of the present invention will employ, unless otherwise indicated, conventional techniques of virology, protein chemistry, cell biology, cell culture, molecular biology, microbiology, and recombinant DNA, which are within the skill of the art. Such techniques are explained fully in the literature. See, for example, Clinical Virology, 2^{nd} Ed., by Richman, Whitley, Hayden (American Society for Microbiology Press: 2002), Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); and Methods In Enzymology, Vols. 154 and 155 (Wu et al. eds.). Cell sorting and cell analysis methods are known in the art and are described in, for example, The Handbook of Experimental Immunology, Volumes 1 to 4, (D. N. Weir, editor) and Flow Cytometry and Cell Sorting (A. Radbruch, editor, Springer Verlag, 1992).

The invention now being generally described, it will be more readily understood by reference to the following examples, which are included merely for purposes of illustration of certain aspects and embodiments of the present invention, and are not intended to limit the invention.

### EXEMPLIFICATION

### Example 1

### CD40L Surface Expression on Antigen-Stimulated Rhesus Macaque CD4+ T cells

A recent report described the identification of antigen-specific human CD4+ T cells based on upregulation of CD40L (CD154) expression (Bitmansour et al., 2001). However, in this study the mean fluorescent intensity (MFI) of CD40L cell surface stained cells appeared significantly lower compared to CD40L intracellular stained cells, suggesting that only a fraction of CD40L in activated human CD4+ T cells at any time is cell surface expressed. To extend and adapt CD40L surface staining for isolating of viable rhesus antigen-specific CD4+ T cells the following experiments were undertaken.

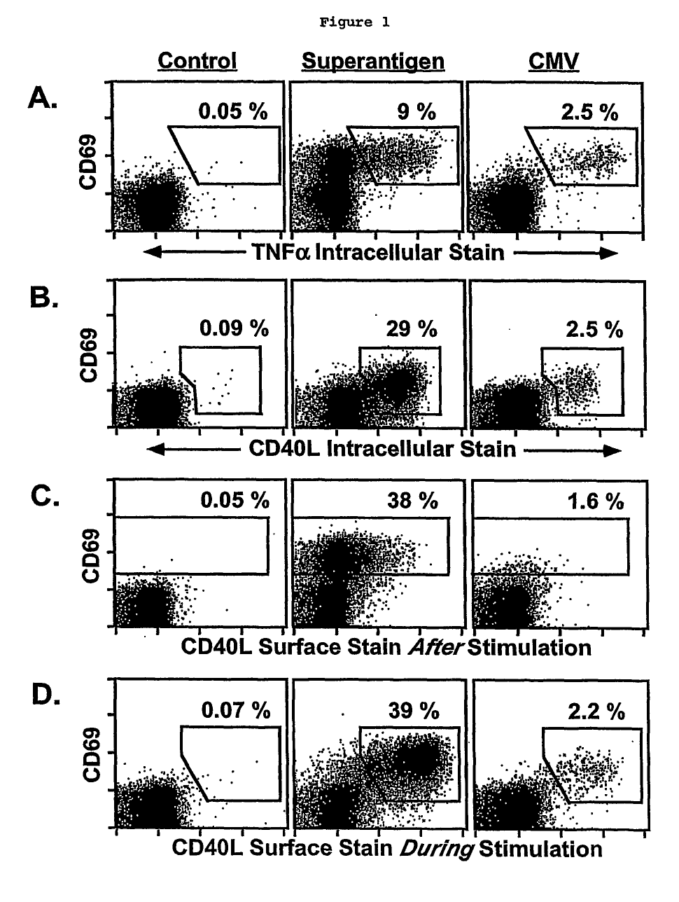

PBMC from rhesus macaques were antigen-stimulated, and the frequencies of CD40L+CD4+ T cells were compared to that of two well-characterized rhesus CD4+ T cell activation markers: TNFα and CD69 (Kaur et al., 2002; Picker et al., 1995). Because TNFα is a secreted molecule, intracellular cytokine staining (ICS) was used in these initial experiments to facilitate comparisons among the three proteins. As shown in FIG. 1, upon stimulation with either superantigen or whole rhesus cytomegalovirus (CMV) antigen, both CD40L and TNFα were upregulated on CD4+CD69+ T cells (FIG. 1A: TNFα; FIG. 1B: CD40L). However, superantigen stimulation (second column) consistently resulted in a lower percentage of CD69+TNFα+ cells than CD69+CD40L+ cells. (FIG. 1A: 9% TNFα+CD69+; FIG. 1B: 29% CD40L+CD69+), suggesting that not all activated CD4+ T cells secreted TNFα during the stimulation. In contrast to superantigen, the frequencies of CMV-specific CD4+ T cells (or SIV-specific CD4+ T cells) identified by TNFα a or CD40L ICS were similar (2.5% FIG. 1A & B, respectively, third column). The disparity observed in the frequency of TNFα+ and CD40L/CD69+ cells following superantigen stimulation underscores the fact that defining antigen-specificity based on a single cytokine may potentially underestimate the frequency and complexity of antigen-specific T cell responses.

In the above analysis, although CD40L expression was upregulated on activated CD4+ T cells (FIG. 1B), its detection in an ICS assay following intracellular retention by Brefeldin A (Picker et al., 1995) does not permit sorting live rhesus T cells based on CD40L surface staining. To address this question, the above experiments were repeated using CD40L surface staining on viable PBMC stimulated by antigen in absence of Brefeldin A. When stained in this manner, although superantigen clearly upregulated CD69 surface expression, low levels of surface CD40L were detected in only a minority of CD69+ cells (FIG. 1C, second column). More importantly, when a specific antigen, either CMV (FIG. 1C, third column) or SIV-specific peptides was employed, CD4+ T cell CD69 surface expression was muted and CD40L surface expression virtually disappeared (FIG. 1C, third column). And while some CMV-specific CD4+ T cells could still be identified based on CD69 surface expression alone, the identification was not robust (indeed, relatively small shifts in the CD69-gate in FIG. 1C decreased the percentage of CMV-specific CD4+ T cells from 1.6% to 0.05%).

The disparity between intracellular and cell surface CD40L staining (FIG. 1B & C) suggests that following antigen activation little of the newly synthesized CD40L accumulated at the cell surface. The simplest explanation of this phenomenon is that most CD40L never reaches the T cell surface. However, this explanation seems unlikely given that the function of T cell CD40L is to bind CD40 expressed on B cells and stimulate T-helper contact-dependent B cell proliferation (Grewal and Flavell, 1998). Therefore, it seemed more likely that the failure to detect cell surface CD40L was not due to a failure of CD40L to reach the cell surface, but rather that upon reaching the cell surface it either undergoes rapid proteolysis (Hirohata, 1999) or endocytosis following binding to its cognate receptor, CD40, (Yellin et al., 1994). Based on the data described herein, the latter explanation, rapid CD40L endocytosis seemed more plausible to us.

CD40L surface staining was therefore redone, this time including the CD40L-PE antibody during the period of antigen stimulation, in the hope that CD40L transiently expressed on the cell surface would be labeled by antibodies prior to endocytosis. As shown in FIG. 1D, staining during stimulation did dramatically enhance CD40L staining (x-axis) of superantigen stimulated CD4+ T cells when compared to cells stained after antigen stimulation (compare FIG. 1C & D, second column, MFI over background increased from 22 to 762, respectively, a 35-fold increase). Moreover, a similar increase was seen in CMV-stimulated CD4+ T cells (FIG. 1C & D, third column, MFI over background 14 to 515, respectively; a 37-fold increase). Qualitatively similar increases were seen in all nine rhesus macaques tested (Table 1: median CD40L MFI increased ˜33-fold and the mean increased ˜16-fold). In contrast to rhesus macaques, for the three human subjects tested, the difference between the two staining techniques resulted in only a 4-fold increase a mean (± standard deviation) increase in CD40L MFI staining after stimulation from 192±31 to 801±78, n=3. The same percentage of activated cells could be identified based on CD40L upregulation regardless of the staining method employed.

TABLE 1
The Effect of Staining After or During Antigen
Stimulation on CD40L Signal Intensity.
CD40L MFI Over Background

| | Stained | Stained | |
|---|---|---|---|
| Rhesus | After | During | Fold- |
| Animal # | Stimulation | Stimulation | Increase |
| 1 | 44 | 452 | 10 |
| 2 | 9 | 427 | 47 |
| 3 | 135 | 920 | 7 |
| 4 | 10 | 447 | 45 |
| 5 | 22 | 762 | 35 |
| 6 | 46 | 889 | 19 |
| 7 | 71 | 657 | 9 |
| 8 | 15 | 731 | 49 |
| 9 | 24 | 784 | 33 |
| Median | 24 | 731 | 33 |

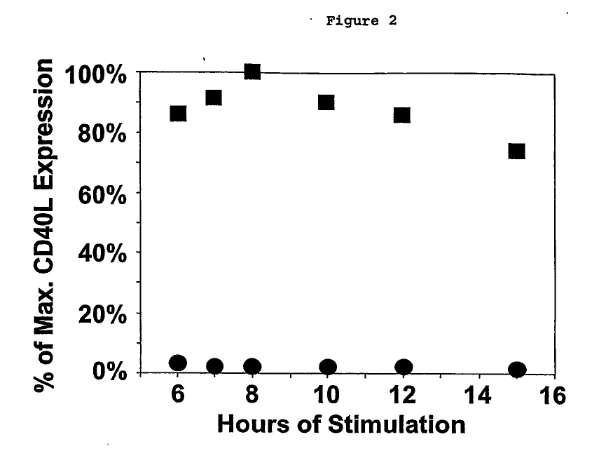

During a time course analysis, maximal CD40L expression was observed 7-9 hours following stimulation when staining occurs during antigen stimulation—although staining intensity only declined <25% when stimulation/staining was extended to 15 hours (FIG. 2). At all time points tested CD40L staining during stimulation was superior to staining after (FIG. 2; additional experiments to optimize CD40L staining during antigen stimulation are described in Example 6).

### Example 2

### Surface Expression of Other Activation Markers on Rhesus CD4+ T cells

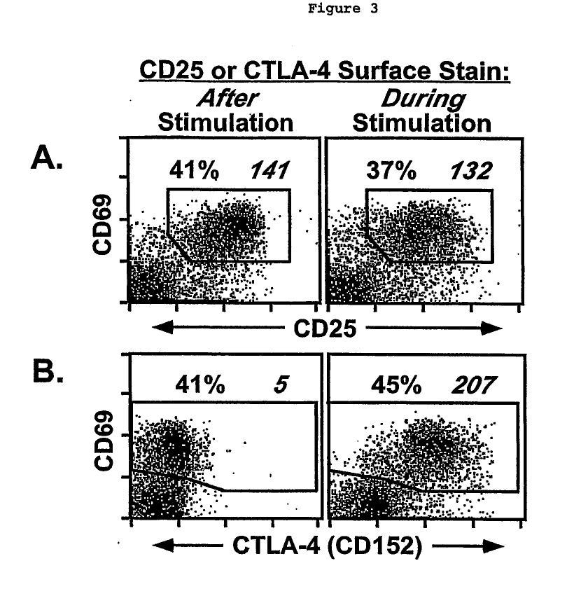

Three other surface proteins expressed on activated CD4+ T cells, CD25, CTLA-4 (CD152), and CD69 were also examined for enhanced surface staining when stained during, as opposed to after, stimulation. Neither the CD25 MFI (141 and 132) nor the percent of positive T cells (41% and 37%) differed significantly when CD25 staining after and during antigen stimulation were compared (FIG. 3A, left and right columns, respectively). CD69 staining was similar to that of CD25 in that T cells stained after stimulation had significant levels of extracellular CD69 (FIG. 3A). And while CD69 staining intensity did increase when staining coincided with stimulation, it did not increase dramatically (<2-fold). In contrast to CD25 and CD69, CTLA-4 staining (FIG. 3B) was significantly affected by the staining method (left and right columns: staining after and during stimulation, respectively, MFI 5 and 207, a ˜40-fold difference).

The above results are consistent with the known cellular trafficking patterns of CD40L, CTLA-4, CD25, and CD69 (Egen et al., 2002; Hemar et al., 1995; Testi et al., 1994; Yellin et al., 1994). For instance, the strong staining seen when CD40L and CTLA-4 are stained during but not after antigen stimulation suggests that both of these molecules are transiently cell surface expressed but subsequently undergo rapid endocytosis and are consistent with previously published reports (Egen et al., 2002; Yellin et al., 1994). By staining during stimulation, CD40L and CTLA-4 transiently expressed on the cell surface can be antibody labeled prior to sequestration from external antibodies.

### Example 3

### Cellular Localization of CD40L and CTLA-4 when Stained in the Presence of Antigen

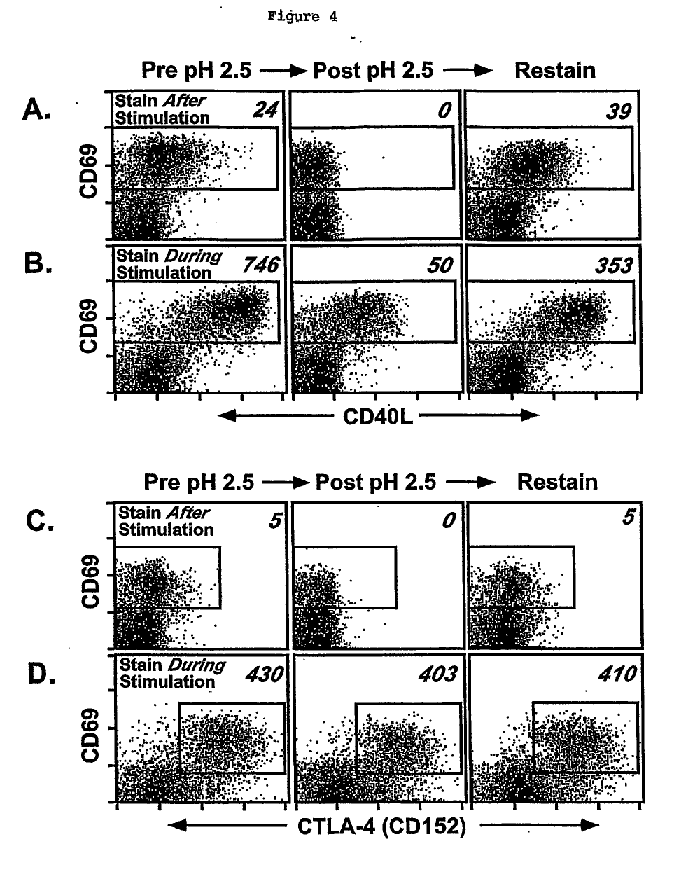

From the above analysis, it is not clear if the presence of antibodies specific for CD40L or CTLA-4 during antigen stimulation prevents endocytosis or if endocytosis proceeds regardless of whether CD40L or CTLA-4 are complexed to antibody. Knowing the cellular localization of CD40L and CTLA-4 and the associated conjugated-antibody is important for determining an appropriate enrichment strategy for antigen-specific T cells. For example, if the PE-conjugated antibody were endocytosed, it would not be accessible to subsequently added extracellular anti-PE magnetic beads. Therefore, to quantify the relative amount of extra- and intracellular CD40L and CTLA-4 in activated CD4+ T cells, stained cells were briefly washed with a low pH (pH 2.5) buffer. This treatment dislodges extracellular antibodies while internalized antibodies remain cell-associated and intact (Means et al., 2002). Control experiments established that when PBMC were stained for either CD40L or CTLA-4 following superantigen stimulation (this staining is done on ice, in the presence of sodium azide and EDTA—conditions that should prevent endocytosis) although staining was as expected weak (FIG. 4A & C, respectively, left-hand column) the CD40L or CTLA-4 staining was acid labile (FIG. 4A & C, respectively, middle column; except for CD40L and CTLA-4 staining, all other staining [i.e. CD3, CD4, and CD69] in FIG. 4 followed antigen stimulation and acid treatment). Furthermore, when the acid washed cells were restained for CD40L and CTTA-4, the weak staining in the CD69-positive population was restored (FIG. 4A & C, respectively, right-hand column). Taken together, these results suggest that when antibodies specific for CD40L and CTLA-4 are added following antigen stimulation, the antibodies remain extracellular and are removed by the low pH wash.

Similar experiments were then performed on cells stained for CD40L and CTLA-4 during antigen stimulation. As expected, prior to low pH washing these cells stained brightly for both CD40L and CTLA-4 (FIG. 4B & D, left-hand column). However, upon low pH washing, disparate results were seen for CD40L and CTLA-4 staining (FIG. 4B & D, middle column). While CD40L staining decreased significantly in intensity, CTLA-4 staining was unaffected (CD40L staining fell 15-fold, from a MFI of 746 to 50; while CTLA-4 MFI only decreased from 430 to 403; the constancy of CTLA-4-PE staining also serves as a control, demonstrating that the PE-fluorophore itself is resist to the low pH wash). Restaining for CD40L following the low pH wash restored much but not all of the CD40L signal (FIG. 4B, right-hand column MFI 353) but, had little effect on CTLA-4 (FIG. 4D, right-hand column, MFI 410). These results suggest that when their respective antibodies are present during antigen stimulation, CD40L largely gets trapped on the cell surface, while CTLA-4 still undergoes endocytosis. These results suggest that the CD40L antibody employed here (TRAP-1) may block CD40/CD40L interactions and thereby prevent CD40L endocytosis. In contrast to CD40L, whose internalization may require binding to CD40 (Yellin et al., 1994), CTLA-4 endocytosis is the default pathway and occurs whether or whether not CTLA-4 is complexed with antibody (Alegre et al., 1996; Egen et al., 2002; Linsley et al., 1996).

### Example 4

### Purification of CD40L+ Cells

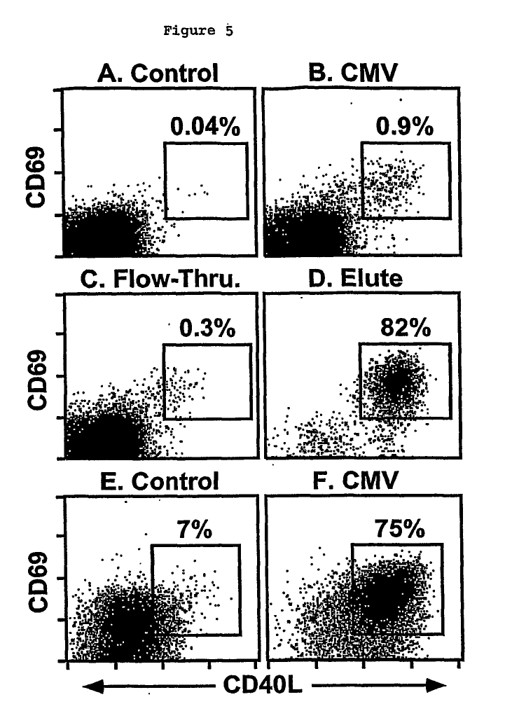

Following stimulation of rhesus PBMC with whole CMV antigen and CD40L staining during the stimulation period, CMV-specific CD4+ T cells were enriched using magnetic beads and/or flow cytometry. FIG. 5, shows typical CMV-specific CD4+ T cell enrichment using magnetic bead purification (FIG. 5A-5D). The CMV-enriched population (FIG. 5D) was then expanded 12 days in culture with feeders and IL-2. On day 12, >99% of the surviving cells were CD4+. Moreover, upon restimulation, ˜75% of the CD4+ T cells were antigen-specific based on CD40L and CD69 expression (FIG. 5E & 5F; control and whole CMV antigen stimulation, respectively).

### Example 5

### Genetic Modification of CD40L+ Cells

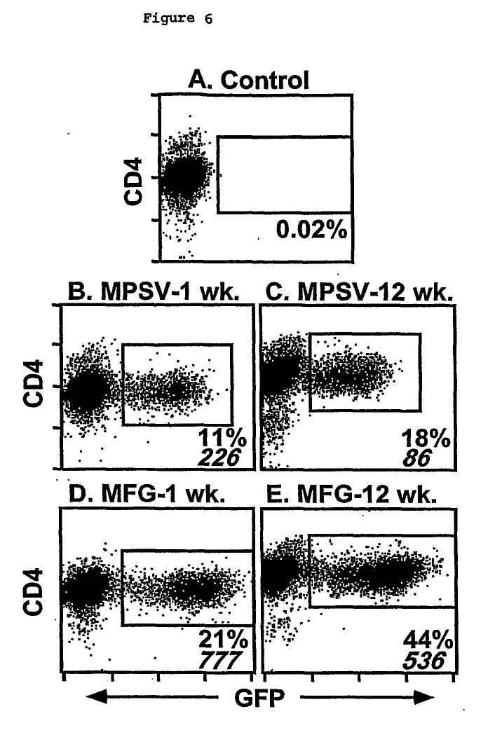

One of the goals of this study was to obtain a method for rapidly isolating and genetically modifying rhesus CD4+ T cells for reconstitution studies. To test the feasibility of genetically modifying CD4+CD40L+ T cells, superantigen stimulated CD4+CD69+CD40L+ T cells were enriched using magnetic beads and fluorescence activated cell sorting, expanded in culture for five days, and transduced with various retroviral vectors encoding for GFP. As shown in FIG. 6, retroviral vectors readily transduced the purified CD4+CD40L+ T cells and slightly better transduction efficiency and GFP expression were obtained with the murine retroviral vector employed (FIG. 6D) than with the lentiviral vectors (FIG. 6B). Similar transduction efficiencies and GFP expression were also seen for CMV stimulated and purified CD40L+CD4+ T cells.

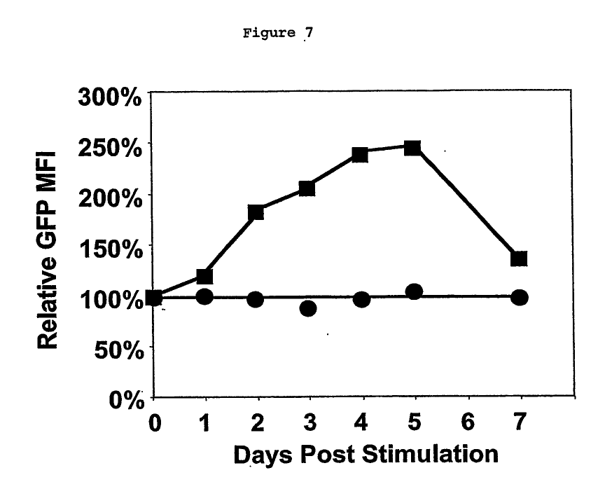

To monitor GFP expression stability, four polyclonal transduced cell lines similar to the one shown in FIG. 6D were kept 12 weeks in culture, with fresh IL-2/media feedings every 3-4 days, and Concanavalin A restimulation every 12-14 days. GFP expression in the MFG cell lines was monitored biweekly during this period. At week one, the mean four GFP MFI of individual cell lines GFP MFI was 717±82 (± standard deviation). Although GFP expression initially declined during the first 3-4 weeks in culture, after that GFP expression stabilized, and at week twelve the cell lines the mean GFP MFI was 443±97 (see FIG. 6D & E for representative week one and twelve flow data). Similar results were noted in lentivirus-transduced cell lines FIG. 6B & C). Repetitive T cell stimulation was not required to maintain GFP expression, since mitogen stimulation led only to a transient, 2-3 fold increase in GFP expression, peaking at day 6, before returning to baseline (FIG. 7). Moreover, even T cell lines maintained in culture for 10-12 weeks without concanavalin A stimulation (these cells were refed with fresh media/lL-2 every few days and did not noticeably expand in culture), GFP expression was about half that of cells maintained with concanavalin A restimulation over the same period. Therefore, although T cell activation does upregulate transgene expression in vitro, it is not strictly required to maintain GFP expression in CD40L+ isolated T cells.

In summary, we have shown that antigen stimulation in the presence of conjugated CD40L antibody significantly enhances CD4+ T cell CD40L surface staining and makes identification of antigen-specific CD4+ T cells based on CD40L staining possible. CD4+ T cells isolated by this technique are viable, rapidly expand in culture, and are readily transduced with retroviral vectors. This methodology should facilitate isolation of RNA from antigen-specific CD4+ T cells and expansion and transduction of antigen-specific CD4+ T cells. In addition, the use of the surface trapping approach may prove useful as a general method for the identification of antigen-specific T cells based on activation markers that do not normally reach high concentrations on the cell surface.

### Example 6

### CD40L/CD69 Upregulation in Central & Effector Memory CD4 T Cell Populations

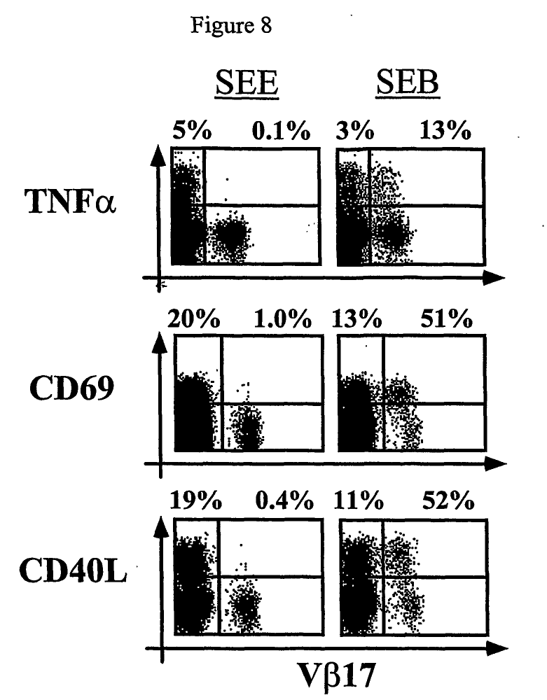

As previously shown, in stimulated CD4 T cells there is poor correlation between CD69 and TNFα upregulation (FIG. 1A, second column). In contrast, there is much better correlation among surface activation markers: CD69, CD40L, CD25, and CTLA-4 (FIGS. 1 & 3). A trivial explanation for this is that surface activation markers are upregulated non-specifically by ‘bystander’ activation while cytokine secretion is not. For example, cytokines alone can lead to T cell proliferation or CD69/CD40L upregulation in absence of TCR signaling. Similarly, T cells from extra-lymphoid tissue may constitutively express CD69 in absence of overt antigen. Therefore, to test whether T cell upregulation of CD40L/CD69 was due to ‘specific’ activation (i.e. through TCR-dependent pathways), we investigated whether superantigen-induced CD40L/CD69 upregulation occurred on specific subsets of T cells consistent with the specificity of the superantigen (for example, SEB specifically activates human T cells bearing Vβ17+ TCR—as well as a few other Vβ families—while SEE is not Vβ17 specific), or whether CD40L/CD69 upregulation was ‘nonspecific’ and not Vβrestricted. As shown in FIG. 8, TNFα, CD40L, and CD69 were only weakly upregulated on Vβ17+ CD4 T cells stimulated with SEE (FIG. 8, left-hand panels, 0.1%, 1.0%, and 0.4%, respectively) but strongly upregulated by SEB (FIG. 8, right-hand panels, 13%, 51%, and 52% respectively). Importantly, both for Vβ7+ and Vβ17-negative cells, for SEB stimulation, a four-fold disparity exists between TNFα+ and CD69+/CD40L+ cells (for Vβ17+ cells the percentages were: 13%, 51%, & 52%, respectively; while for Vβ17-negative cells the percentages were: 3%, 13%, & 11%). These results argue that the vast majority of CD69+ or CD40L+ CD4 T cells have been activated ‘specifically’ through TCR-dependent pathways.

Therefore, a more likely explanation for the failure of most activated CD4 T cells to secrete TNFα is that the capacity to secrete TNFα is differentiation state dependent and increases as follows: naïve T cells <central memory T cell (TCM) <effector memory T cell (TEM). In contrast, the expression of CD69 is not differentiation state dependent. We therefore investigated whether isolation of antigen-specific CD4 T cells based on cytokine secretion preferentially isolated TEM cells but failed to detect many antigen-specific naïve and TCM cells.

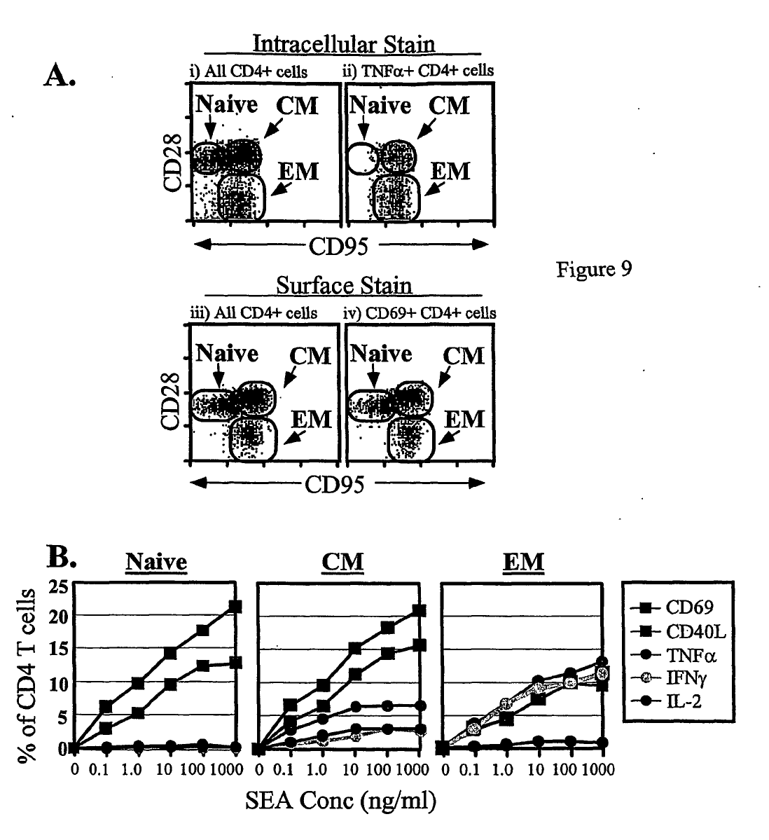

The classification of rhesus macaque peripheral blood CD4 T cells as naïve, TCM, or TEM is based on CD28 and CD95 staining. In FIG. 9A, for superantigen stimulated rhesus PBMC, we show the CD28-CD95 staining profile for: (1) all CD4 T cells (FIG. 9Ai) or (2) only TNFα+ CD4 T cells (FIG. 9Aii). Strikingly, but not unexpectedly, naïve CD4 T cells were virtually excluded from the TNFα+ population. In contrast, the CD69+ population contained many naïve CD4 T cells (FIG. 9A iv).

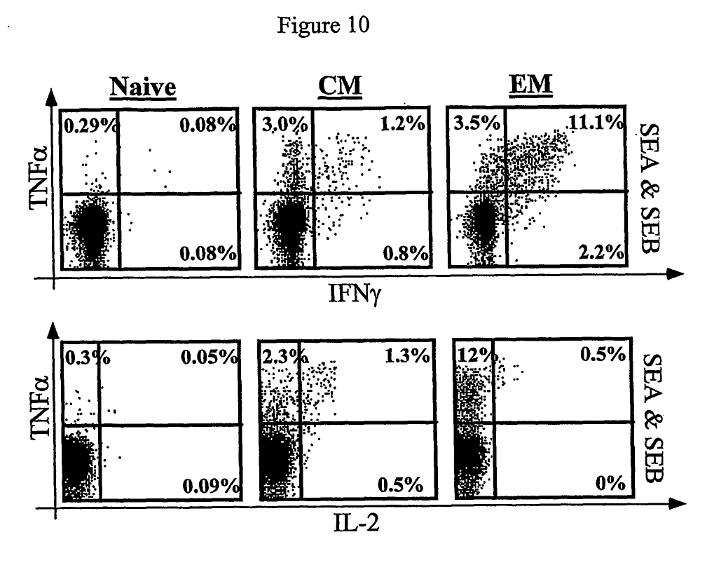

To more rigorously quantify these differences, the above analysis was extended to two other cytokines, IFNγ and IL-2, and the concentration of superantigen varied over 4 orders of magnitude. As shown in FIG. 9B (left column), naïve macaque CD4 T cells upregulated CD40L and CD69 but did not make significant amounts of TNFα, IFNγ, or IL-2. More significantly, although TCM cells secreted TNFα and IFNγ, they did so only in a minority of the CD69+ T cell population (FIG. 9B, middle column; in the 7 monkeys tested, TNFα secretion was on average ˜25% that of CD69 upregulation while IFNγ secretion was ˜10-15% that of CD69). These disparities did not extend to the TEM population where the frequency of TNFα+, IFNγ+, and CD69+ cells were similar (FIG. 9B, right hand column). However, in contrast to their relative abilites to secrete TNFα and IFNγ, TCM cells consistently had an equal if not greater capacity to secrete IL-2 than did TEM cells (FIG. 9B). These results are more dramatically visualized when one examines dot-plots for cytokine production (FIG. 10). For example, the poor correlation between TNFα and IFNγ production is apparent in the TCM population, but not in the TEM population (FIG. 10, top row, middle and right-hand columns, respectively). In contrast, a disproportionately greater percentage of TCM cells are TNFα/IL-2 double positive than are TEM cells (FIG. 10, bottom row, one in three TCM cells were INFα+IL2+, while one in twenty-five TEM cells were TNFα+IL-2+). The greater propensity of TCM cells to secrete IL2 is consistent with their greater proliferative capacity.

Thus, we have shown that memory CD4 T cells isolated based on CD40L surface trapping are more representative of the actual in vivo mix of CD4 TCM and TEM cells than are CD4 T cells isolated based on cytokine secretion.

### Example 7

### Materials and Methods for Examples 1-6

Intracellular Cytokine Staining and CD40L Upregulation Assays

PBMC were isolated from 10-20 ml rhesus blood by Ficoll-Paque (Pharmacia, San Diego, Calif.). Initial experiments indicated that freezing PBMC altered the recovery of antigen-specific CD4+ T cells by <30%, therefore, both fresh and frozen PBMC were used in this study. PBMC were stimulated with either whole rhesus CMV antigen (50-150 μg/tube (Kaur et al., 2002) or superantigen, Staphylococcal enterotoxin A and B (100 ng/ml each; Sigma, St. Louis, Mo.), in the presence of anti-CD28 and anti-CD49d antibodies (BD Biosciences, San Diego, Calif.) cross-linked by goat-anti-mouse antibody (Kierkegaard Perry Labs, Gaithersburg, Md.) and intracellular cytokine stained as described (Kaur et al., 2002; Picker et al., 1995).

The following antibodies were used for ICS and cell surface staining: CD4-PerCp, CD4-APC, CD3-FITC, CD3-PE, CD8-PE, CD69-PE, CD69-APC, CD25-PE, CTLA-4 (CD152)-PE, TNFα-APC (BD Biosciences). CD40L-FITC, -APC, and -PE were from TRAP-1 clones (Coulter, Miami, Fla. or BD Biosciences). For cell surface staining during the stimulation period (done at 37° C. in a CO₂ incubator), sodium azide was removed from the antibody preparation prior to staining by multiple rounds of antibody dilution and microconcentration (at least three rounds were performed), whereby: 100-300 μl of antibody was diluted to 500 μl with PBS (Cellgro, Herndon, Va.), concentrated to 50-100 μl in a Microcon YM30 concentrator (Millipore, Bedford, Mass.), and diluted back to 500 μl with PBS. After the last concentration, RPMI media (Sigma) containing 10% fetal calf serum supplemented with glutamine, penicillin/streptomycin, and 10 mM Hepes, pH 7.2 (Cellgro; henceforth, referred to as R-10 media), was added restoring the antibody to its original volume. Azide-free antibody preparations were made on the day of use. 10-20 μl of azide-free antibody was used per 100 μl of PBMC. PBMC were at 2 to 40 million cells per ml in 0.1 to 1.0 ml of R-10 media in 5 ml polystyrene tubes (Falcon/Becton Dickinson, Franklin Lakes, N.J.). Tubes were slanted at ˜5° above horizontal during stimulation as described (Picker et al., 1995). Azide-free antibody was added 1-2 hours following antigen addition. Following stimulation/staining, PBMC were washed with ice cold PBS, 1% fetal calf serum, 2 mM EDTA, 0.02% sodium azide and stained on ice for other cell surface markers.

Additional experiments determined that to optimize CD40L staining during antigen stimulation: (1) For an 8-hour superantigen stimulation, as the length of time the antibody was present during stimulation increased from: 2, 3, 4, 5, to 6 hours (i.e. the antibody was added during the last 2, 3, 4, 5, and 6 hours of the 8 hour stimulation) CD40L MFI increased from: 162, 236, 295, 381, to 427, respectively. (2) Staining for CD40L after antigen stimulation in addition to staining during antigen stimulation did not increase CD40L surface staining. (3) Staining during stimulation enhanced CD40L staining for all CD40L antibody conjugates tested (FITC, PE, and APC) but the PE conjugate best separated CD40L-positive from negative cells and all results reported herein are based on CD40L-PE conjugate.

Acid Stripping of Antibody-Stained Cells

Cells were stained with CD40L or CTLA4 PE-conjugated antibodies either during or after antigen stimulation. Stained cells were subsequently centrifuged at 4° C., aspirated to remove as much liquid as possible, placed on ice, and 200 μl of ice-cold 150 mM Sodium Phosphate pH 2.5 was added for 1 minute. Control experiments established that this technique was nearly ˜100% efficient at removing conjugated antibodies specific for cell surface membrane proteins (e.g. CD3 and CD8). After one minute, four ml of ice cold R-10 media containing 25 mM HEPES pH 7.2 (Cellgro) was added to neutralize the acid. The cells were then centrifuged and restained for other surface markers (CD3, CD4, CD69 and, where indicated, restained for CD40L or CTLA-4).

Generation of T Cell Lines

PBMC were stained for CD40L expression during antigen stimulation, washed, and stained for CD3, CD4, and CD69 as described herein. Magnetic enrichment of CD40L-PE+ cells was performed using anti-PE magnetic beads purchased from Miltenyi Biotec (Auburn, Calif.) and followed the manufacturer's protocol with the following modifications. Two rounds of binding and elution of cells to the magnetic column were done. When purifying CMV-specific CD4+ T cells, to minimize the loss of the PE-positive cells during subsequent steps (e.g. centrifugation) all elutions from the magnetic column were done in the presence of 2-5 million irradiated (unstained) human ‘carrier’ feeder cells. Because of the presence of the unstained carrier cells it is difficult to determine the eluted fraction's T cell purity because irradiated feeders as well as rhesus contaminating B and NK cells are all CD3, CD4, CD40L, CD69 negative. However, following 2 weeks in culture, during which time the irradiated feeder cells die, the cultured cells were generally, >97% CD3+ CD4+. Nevertheless, despite the starting purity, given extended time in culture outgrowth of CD4-negative cells was observed (e.g., compare FIG. 6B and 6C, at 1 and 12 weeks, respectively).

CD40L+ enriched cells were grown in R-10 media containing 2-3×10⁶ irradiated (3,000 rads; 30 Gy) human feeder cells/ml in 24- and 48-well plates. Recombinant human IL-2 (50 units/ml; Hoffmann La-Roche, Nutley, N.J.) was added 2-3 days following enrichment and thereafter cells were fed R-10/IL-2 media every 3-4 days. For long-term culture, cell lines were restimulated with concanavalin A (5 μg/ml; Sigma) and irradiated feeder cells every 12-14 days.

T cell lines were tested for antigen specificity 10-14 days after isolation (when most irradiated feeder cells had died) as follows. T cell lines were mixed with autologous B cells pulsed with whole CMV antigen (relatively high B to T cell ratios, 2:1 to 10:1, were needed for maximal activation of T cell lines). T cells were assessed for antigen specificity based on flow analysis of CD69, TNFα, or CD40L ICS expression. The percentage of CD4+ T cells that were scored antigen-specific in T cell lines varied between 25%˜80%.

Retrovirus Production & Transduction

The amphotropic murine leukemia virus vector LZRS/eGFP, which expresses eGFP under the control of the native LTR, was produced as previously described (Rosenzweig et al., 2001). An HIV-1 lentiviral vectors (HRST-MPSV-eGFP-ST) encoding for enhanced GFP and pseudotyped with VSV-G envelope were obtained from the Harvard Gene Therapy Initiative (httD://hgtti.med.harvard.edu). These constructs are self-inactivating (SIN) vectors (Miyoshi et al., 1998; Yu et al., 1986) with GFP expression driven by the MPSV promoters. Lentiviral stocks were titered on U20S cells (American Type Culture Collection) at ˜3×10⁷ units/ml and murine retroviral vectors were concentrated to similar titers using a Centriprep YM-30 filter (Millipore) as described (Rosenzweig et al., 2001).

CD4+ T cell transduction with retroviral vectors was carried out as follows. Five or six days following purification of CD4+CD40L+ T cells (enough time to allow for the death of most feeder cells) 100,000 to 200,000 target T cells in R-10/IL-2 media were mixed with vector supernatants at an MOI of 1-3 u/cell in 24-well non-tissue culture plates (Falcon/Becton Dickinson) pretreated with 25 μg Rectronectin (BioWhittaker, Walkersville, Md.) as described (Pollok et al., 1998). The cells and virus were incubated overnight at 37° C. in a CO₂ incubator. Half the R-10/IL-2 media was exchanged the next day when the cells were restimulated with Concanavalin A and irradiated human feeder cells.

### REFERENCES

Abbas, A. K., Murphy, K. M., and Sher, A. (1996). Functional diversity of helper T lymphocytes., Nature 383, 787-795.
Alegre, M. L., Noel, P. J., Eisfelder, B. J., Chuang, E., Clark, M. R., Reiner, S. L., and Thompson, C. B. (1996). Regulation of surface and intracellular expression of CTLA4 on mouse T cells, J Immunol 157, 47624770.
Altman, J. D., Moss, P. A. H., Goulder, P. J. R., Barouch, D. H., McHeyzer-Williams, M. G., Bell, J. I., McMichael, A. J., and Davis, M. M. (1996). Phenotypic analysis of antigen-specific T lymphocytes., Science 274, 94-96.
Anonymous (1996). The Institute of Laboratory Animal Resources, National Research Council: Guide for the Care and Use of Laboratory Animals., Washington, DC, National Institutes of Health, 86-123.
Bitmansour, A. D., Waldrop, S. L., Pitcher, C. J., Khatamzas, E., Kem, F., Maino, V. C., and Picker, L. J. (2001). Clonotypic structure of the human CD4+ memory T cell response to cytomegalovirus, J Immunol 167, 1151-1163.
Butcher, E. C., and Picker, L. J. (1996). Lymphocyte homing and homeostasis, Science 272, 60-66.
Castle, B. E., Kishimoto, K, Stearns, C., Brown, M. L., and Kehry, M. R. (1993). Regulation of expression of the ligand for CD40 on T helper lymphocytes, J Immunol 151, 1777-1788.
Dal Porto, J., Johansen, T. E., Catipovic, B., Parfiit, D. J., Tuveson, D., Gether, U., Kozlowski, S., Fearon, D. T., and Schneck, J. P. (1993). A soluble divalent class I major histocompatibility complex molecule inhibits alloreactive T cells at nanomolar concentrations, Proc Natl Acad Sci U S A 90, 6671-6675.
Egen, J. G., Kuhns, M. S., and Allison, J. P. (2002). CTLA-4: new insights into its biological function and use in tumor immunotherapy, Nat Immunol 3, 611-618.
Grewal, I. S., and Flavell, R. A. (1998). CD40 and CD154 in cell-mediated immunity, Annu Rev Immunol 16, 111-35.
Hackett, C. J., and Sharma, O. K. (2002). Frontiers in peptide-MHC class II multimer technology, Nat Immunol 3, 887-889.
Hemar, A., Subtil, A., Lieb, M., Morelon, E., Hellio, R., and Dautry-Varsat, A. (1995). Endocytosis of interleukin 2 receptors in human T lymphocytes: distinct intracellular localization and fate of the receptor alpha, beta, and gamma chains, J Cell Biol 129, 55-64.
Hirohata, S. (1999). Human Th1 responses driven by IL-12 are associated with enhanced expression of CD40 ligand, Clin Exp Immunol 115, 78-85.
Kaur, A., Hale, C. L., Noren, B., Kassis, N., Simon, M. A., and Johnson, R. P. (2002). Decreased frequency of cytomegalovirus (CMV)-specific CD4+ T lymphocytes in simian immunodeficiency virus-infected rhesus macaques: inverse relationship with CMV viremia, J Virol 76, 3646-3658.
Lanzavecchia, A., and Sallusto, F. (2000). Dynamics of T lymphocyte responses: intermediates, effectors, and memory cells, Science 290, 92-97.
Linsley, P. S., Bradshaw, J., Greene, J., Peach, R., Bennett, K. L., and Mittler, R. S. (1996). Intracellular trafficking of CTLA-4 and focal localization towards sites of TCR engagement, Immunity 4, 535-543.
Masuda, N., Ohnishi, T., Kawamoto, S., Monden, M., and Okubo, K. (1999). Analysis of chemical modification of RNA from formalin-fixed samples and optimization of molecular biology applications for such samples, Nucleic Acids Res 27, 4436-4443.
McKay, P. F., Schmitz, J. E., Barouch, D. H., Kuroda, M. J., Lifton, M. A., Nickerson, C. E., Gorgone, D. A., and Letvin, N. L. (2002). Vaccine protection against functional CTL abnormalities in simian human immunodeficiency virus-infected rhesus monkeys, J Immunol 168, 332-337.
Means, R. E., Choi, J. K., Lee, B. S., and Jung, J. U. (2002). Multiple endocytic trafficking pathways of MHC class I molecules induced by a Herpesvirus protein, J Virol 76, 4688-4698.
Miyoshi, H., Blomer, U., Takahashi, M., Gage, F. H., and Verma, I. M. (1998). Development of a self-inactivating lentivirus vector, J Virol 72, 8150-8157.
Picker, L. J., Singh, M. K., Zdraveski, Z., Treer, J. R., Waldrop, S. L., Bergstresser, P. R., and Maino, V. C. (1995). Direct demonstration of cytokine synthesis heterogeneity among human memory/effector T cells by flow cytometry., Blood 86, 1408-1419.
Pollok, K. E., Hanenberg, H., Noblitt, T. W., Schroeder, W. L., Kato, I., Emanuel, D., and Williams, D. A. (1998). High-efficiency gene transfer into normal and adenosine deaminase-deficient T lymphocytes is mediated by transduction on recombinant fibronectin fragments, J Virol 72,4882-4892.
Rosenzweig, M., Connole, M., Glickman, R., Yue, S.-P. S., Noren, B., DeMaria, M., and Johnson, R. P. (2001). Induction of cytotoxic T lymphocyte and antibody responses to enhanced green fluorescent protein following transplantation of transduced CD34+ hematopoietic cells., Blood 97, 1951-1959.
Testi, R., D'Ambrosio, D., De Maria, R., and Santoni, A. (1994). The CD69 receptor: a multipurpose cell-surface trigger for hematopoietic cells, Immunol Today 15, 479-483.
Welsh, R. M. (2001). Assessing CD8 T cell number and dysfunction in the presence of antigen, J Exp Med 193, F19-22.
Whitmire, J. K., and Ahmed, R. (2001). The economy of T-cell memory: CD4+ recession in times of CD8+ stability?, Nat Med 7, 892-893.
Yellin, M. J., Sippel, K., Inghirami, G., Covey, L. R., Lee, J. J., Sinning, J., Clark, E. A., Chess, L., and Lederman, S. (1994). CD40 moleculaes induce down-modulation and endocytosis of T cell surface T cell-B cell activating molecule/CD40-L. Potential role in regulating helper effector function., J Immunol 152, 598-608.
Yu, S. F., von Ruden, T., Kantoff, P. W., Garber, C., Seiberg, M., Ruther, U., Anderson, W. F., Wagner, E. F., and Gilboa, E. (1986). Self-inactivating retroviral vectors designed for transfer of whole genes into mammalian cells, Proc Natl Acad Sci U S A 83, 3194-8.
Cardin, R. D., J. W. Brooks, S. R. Sarawar and P. C. Doherty (1996). “Progressive loss of CD8+ T cell-mediated control of a gamma-herpesvirus in the absence of CD4+ T cells.” J. Exp. Med. 184(3): 863-71.
Dittmer, U., W. Luke, C. Stahl-Hennig, C. Coulibaly, H. Petry, W. Bodemer, G. Hunsmann and G. Voss (1994). “Early helper T-cell dysfunction in simian immunodeficiency virus but not in human immunodeficiency virus type-2-infected macaques.” J. Med. Primatol. 23: 298-303.
Kalams, S. A. and B. D. Walker (1998). “The critical need for CD4 help in maintaining effective cytotoxic T lymphocyte responses.” J. Exp. Med. 188(12): 2199-204.
Matloubian, M., R. J. Concepcion and R. Ahmed (1994). “CD4+ T cells are required to sustain CD8+ cytotoxic T-cell responses during chronic viral infection.” J. Virol. 68(12): 8056-63.
Rosenberg, E. S., J. M. Billingsley, A. M. Caliendo, S. L. Boswell, P. E. Sax, S. A. Kalams and B. D. Walker (1997). “Vigorous HIV-1-specific CD4+ T cell responses associated with control of viremia.” Science 278: 1447-1450.
Wahren, B., L. Morfeldt-M\dot{a}nsson, G. Biberfeld, L. Moberg, A. Sönnerborg, Pljungman, A. Werner, P. Kurth, R. Gallo and D. Bolognesi (1987). “Characteristics of the specific cell-mediated immune response in human immunodeficiency virus infection.” J. Virol. 61: 2017-2023.
Walter, E. A., P. D. Greenberg, M. J. Gilbert, R. J. Finch, K. S. Watanabe, E. D. Thomas and S. R. Riddell (1995). “Reconstitution of cellular immunity against cytomegalovirus in recipients of allogeneic bone marrow by transfer of T-cell clones from the donor.” N. Engl. J. Med. 333: 1038-1044.
Zajac, A. J., J. N. Blattman, K Murali-Krishna, D. J. Sourdive, M. Suresh, J. D. Altman and R. Ahmed (1998). “Viral immune evasion due to persistence of activated T cells without effector function.” J. Exp. Med. 188(12): 2205-13.
   Equivalents

Those skilled in the art will recognize, or be able to ascertain using no more than routine experimentation, many equivalents to the specific embodiments of the invention described herein. Such equivalents are intended to be encompassed by the following claims.

1. A method for isolating a T cell population, comprising
i. contacting a population of cells comprising a T cell with a first activator that binds to a T cell receptor on the T cell thereby activating the T cell and a first agent that binds to a first cell surface molecule on the T cell, to obtain a T cell population bound by the first agent; and
ii. isolating the T cell population by a method using the first agent,
to thereby isolate a T cell population.

2. The method of claim 1, wherein the T cell is a CD4+ T cell.

3. The method of claim 1, wherein the first cell surface molecule is an activation marker.

4. The method of claim 3, wherein the activation marker is CD40L or CTLA-4.

5. The method of claim 1, wherein the first agent is an antibody or portion thereof sufficient for binding specifically to the surface molecule.

6. The method of claim 1, wherein the first agent is labeled.

7. (canceled)

8. (canceled)

9. The method of claim 6, wherein the method using the first agent comprises using fluorescence activated cell sorting (FACS) or a solid surface to which the T cell binds.

10. (canceled)

11. The method of claim 1, further comprising contacting the T cell population with a first detection agent that specifically binds to the first agent.

12. (canceled)

13. The method of claim 1, wherein the first activator binds to the antigen-binding region of the T cell receptor.

14. The method of claim 13, wherein the first activator is an antigen.

15. The method of claim 1, wherein the first activator does not bind to the antigen-binding region of the T cell receptor.

16. The method of claim 15, wherein the first activator is a superantigen or a polyclonal activator.

17. (canceled)

18. The method of claim 14, wherein the antigen is located on an antigen presenting cell.

19. (canceled)

20. (canceled)

21. (canceled)

22. The method of claim 21, wherein the population of cells comprises peripheral blood mononuclear cells or bone marrow cells.

23. (canceled)

24. The method of claim 1, wherein the population of T cells is contacted essentially simultaneously with the first agent and the first activator.

25. The method of claim 1, wherein the population of T cells is contacted with the first agent prior to being contacted with the first activator, wherein the T cell is contacted simultaneously with the first activator and the first agent for at least about 10 minutes.

26. The method of claim 1, wherein the population of T cells is contacted with the first activator prior to being contacted with the first agent, wherein the T cell is contacted simultaneously with the first activator and the first agent for at least about 10 minutes.

27. The method of claim 1, further comprising contacting the population of T cells with the first agent after contacting the T cell with the first activator.

28. The method of claim 1, further comprising
i. contacting the T cell population with (a) a second activator that binds to the T cell receptor on at least some cells of the T cell population thereby activating at least some cells of the T cell population and (b) a second agent that binds to a second cell surface molecule of at least some cells of the T cell population, to obtain a T cell population bound by the second agent; and
ii. isolating the T cell population by a method using the second agent,
to thereby isolate a T cell population.

29. (canceled)

30. The method of claim 28, wherein the second activator is different from the first activator.

31. (canceled)

32. The method of claim 28, wherein the second agent is different from the first agent.

33. (canceled)

34. The method of claim 32, wherein the second cell surface molecule is different from the first cell surface molecule.

35. The method of claim 34, wherein the first cell surface molecule is CD40L and the second cell surface molecule is CTLA-4.

36. An isolated viable cell population, wherein at least about 90% of the cell population consists of viable CD4+ T cells.

37. (canceled)

38. The isolated viable cell population of claim 36, wherein at least about 90% of the cell population consists of viable CD40L+ CD4+ T cells or CTLA-4+ CD4+ T cells.

39. (canceled)

40. An isolated viable T cell population isolated by the method of claim 1.

41. (canceled)

42. (canceled)

43. A method for treating a subject having cancer or an infectious disease, comprising
(i) obtaining a population of cells comprising a T cell from the subject;
(ii) subjecting the population of cells to the method of claim 1 to thereby obtain a T cell population; and
(iii) administering the T cell population to the subject, to thereby treat the subject having cancer or an infectious disease.
